(19) United States
(12) Patent Application Publication
(10) Pub. No.: US 2025/0322925 A1
(43) Pub. Date: Oct. 16, 2025

(54) METHODS OF MANAGING EOSINOPHILIC ESOPHAGITIS

(71) Applicant: Ellodi Pharmaceuticals, L.P., Lawrenceville, NJ (US)

(72) Inventor: Brian A. MELTZER, Wilton, CT (US)

(21) Appl. No.: 18/967,539

(22) Filed: Dec. 3, 2024

Related U.S. Application Data
(63) Continuation of application No. 18/641,910, filed on Apr. 22, 2024, now abandoned.
Application No. 18/641,910 is a continuation of application No. 16/968,711, filed on Aug. 10, 2020, now abandoned.
(60) Provisional application No. 62/633,432, filed on Feb. 21, 2018.

(51) Int. Cl. G16H 20/10; A61B 5/00; A61B 10/04; A61K 31/4439; A61K 31/575; A61K 31/58; C07K 16/24; C07K 16/28; G16H 10/20

(52) U.S. Cl. CPC G16H 20/10; A61B 5/411; A61B 5/4205; A61B 5/4233; A61B 5/4824; A61B 5/4842; A61B 5/4848; A61K 31/4439; A61K 31/575; A61K 31/58; C07K 16/244; C07K 16/247; C07K 16/2866; G16H 10/20; A61B 10/04; C07K 2317/21; C07K 2317/24

(57) ABSTRACT

Described herein are methods of managing eosinophilic esophagitis (EoE) in a patient, wherein the patient records the number of episodes of dysphagia on a patient report outcome (PRO) questionnaire prior to and during the course of treatment. While on treatment, the number of episodes of dysphagia are reduced, as reported on the PRO questionnaire. In some embodiments, the esophageal eosinophils are counted before or after treatment, or both. In some embodiments, esophageal eosinophils counts are not correlated with a reduction in the episodes of dysphagia.

### CROSS-REFERENCE TO RELATED APPLICATIONS

This application is a continuation of U.S. application Ser. No. 18/641,910, filed Apr. 22, 2024, which is a continuation of U.S. application Ser. No. 16/968,711, filed Aug. 10, 2020, now abandoned, which is the U.S. National Stage of International Patent Application No. PCT/US2019/019040, filed Feb. 21, 2019, which claims the benefit of priority to U.S. Provisional Application No. 62/633,432, filed Feb. 21, 2018, the entire contents of which are hereby incorporated by reference for all purposes.

### BACKGROUND

Dysphagia (e.g. difficulty or pain swallowing) is implicated in a number of diseases or disorders including Achalasia, Diffuse spasm, esophageal stricture (e.g. a narrowed esophagus caused by scar tissue or inflammation), esophageal tumors, esophageal ring, GERD, Scleroderma, and inflammation disorders.

Esophageal inflammation disorders such as eosinophilic esophagitis (EoE), a disease characterized by high levels of eosinophils in the esophagus, as well as basal zonal hyperplasia, is increasingly being diagnosed in children and adults. Many aspects of the disease remain unclear including its etiology, natural history, and optimal therapy. EoE affects all age groups but most frequently individuals between 20 and 50 years of age. Symptoms of EoE often mimic those of gastroesophageal reflux disease (GERD) and include vomiting, dysphagia, pain and food impaction. The disease is painful, leads to difficulty swallowing, and predisposes patients to other complications. EoE is often misdiagnosed for GERD, causing delay in adequate treatment for EoE patients.

Because the symptoms of EoE overlap with GERD and other inflammatory conditions, diagnosis of EoE and treatment is difficult. Currently, EoE is diagnosed by taking esophageal biopsies and finding of 15 or more eosinophils per high power field (HPF). However, another distinguishing feature is dysphagia, since elevated levels of eosinophils can lead to can lead to esophageal fibrosis resulting in loss of esophageal function and the occurrence of dysphagia.

There exists a need in the art for accurate methods of recording episodes of dysphagia and treating dysphagic diseases such as EoE based on thereon.

### SUMMARY OF THE INVENTION

In some embodiments, provided herein is a method of managing eosinophilic esophagitis, in a patient in need thereof, comprising:
(i) prior to treatment with a therapeutic agent,
   (a) recording each episode of dysphagia, at the time the episode occurs, for a period of two weeks using a patient-reported outcome (PRO) questionnaire; and
   (b) measuring esophageal eosinophils in the patient; then
(ii) treating the patient with a therapeutically effective amount of a therapeutic agent for at least two weeks; and
(iii) recording, using the PRO questionnaire, each episode of dysphagia, at the time each episode occurs, while the patient is being treated,
   wherein the number of episodes of dysphagia over any two week period of time while the patient is being treated is reduced compared to the number of episodes of dysphagia prior to treatment.

In some embodiments, the method of managing eosinophilic esophagitis (EoE) in a patient in need thereof, comprises:
(i) prior to treatment with a therapeutic agent, recording each episode of dysphagia, at the time each episode occurs, for a period of two weeks using a patient reported outcome (PRO) questionnaire; then
(ii) treating the patient with a therapeutically effective amount of a therapeutic agent for at least two weeks;
(iii) recording, using the PRO questionnaire, each episode of dysphagia, at the time each episode occurs, while the patient is being treated; and
(iv) measuring esophageal eosinophils in the patient,
   wherein the number of episodes of dysphagia over any two week period of time while the patient is being treated is reduced compared to the number of episodes of dysphagia prior to treatment.

In some embodiments, the therapeutic agent is a corticosteroid, a proton pump inhibitor (PPI), or an antibody, e.g., any therapeutic agent described herein. In some embodiments, the corticosteroid is budesonide, fluticasone, flunisolide, ciclesonide, mometasone, beclomethasone, or tixocortol, or a salt, ester, solvate, polymorph, or prodrug thereof. In some embodiments, the PPI is omeprazole, lansoprazole, dexlansoprazole, esomeprazole, pantoprazole, or rabeprazole. In some embodiments, the antibody is an IL-4, IL-5, or IL-13 antibody. In some embodiments, the antibody is benralizumab, mepolizumab, dupilumab, RPC-4046.

In some embodiments, the recording comprises recording one or more of: (a) incidence of an episode of dysphagia; (b) duration of dysphagia, (c) severity of dysphagia; (d) pain caused by dysphagia; (e) discomfort of dysphagia; and (f) time and date of administering treatment. In some embodiments, the method further comprises scoring the at least one severity question from 0 to 10; scoring the at least one pain question from 0 to 10; scoring the at least one discomfort question from 0 to 10.

In some embodiments, the esophageal eosinophils are measured before treatment, during treatment, or a combination thereof. In some embodiments, esophageal eosinophils are measured by obtaining a biopsy. In some embodiments, the biopsy is an endoscopy. In some embodiments, the method comprise measuring esophageal eosinophils in the patient after the patient has been treated with the therapeutic agent for at least two weeks.

In some embodiments, the patient has an esophageal eosinophil count of ≥15 per high-power field (HPF) prior to treatment.

In some embodiments, the patient is a histological non-responder. In some embodiments, the patient has an esophageal eosinophil count of ≥15 per high-power field (HPF) after treatment.

In some embodiments, the patient has an esophageal eosinophil count of <15 per high-power field (HPF) after treatment. In some embodiments, the patient has an esophageal eosinophil count of ≤6per high-power field (HPF).

In some embodiments, after the patient experiences a reduction in dysphagia, the patient continues treatment with the therapeutic agent at the same dose as used in step. In some embodiments, the method further comprises administering a dose of the therapeutic agent which is decreased by at least about 5%, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, and about 100%.

In some embodiments, the patient is treated with the decreased dose of the therapeutic agent for at least the period of time during which the number of episodes of dysphagia are reduced. In some embodiments, if the number of episodes of dysphagia increases while the patient is receiving the decreased dose, the method further comprises administering a non-reduced dose.

In some embodiments, the patient was not responsive to a PPI.

In some embodiments, prior to treatment, the patient experienced at least three episodes of dysphagia a week for at least two weeks, e.g., 4, 5, 6, 7, 8, 9, 10, 11, 12, 13, 14, 15, 16, 17, 18 19, 20, or more episodes.

In some embodiments, the number of episodes of dysphagia is reduced by at least two episodes, e.g., by 3, 4, 5, 6, 7, 8, 9, 10, 11, 12, 13, 14, 15, 16, 17, 18 19, 20, or more episodes.

In some embodiments, prior to treatment with the therapeutic agent, the patient reported 2 or more episodes of dysphagia per day using the PRO questionnaire. In some embodiments, the number of episodes of dysphagia recorded on the PRO questionnaire is not significantly affected by behavioral modifications. In some embodiments, the behavior modification comprises limiting intake of difficult-to-swallow foods. In some embodiments, the episodes of dysphagia are recorded by food type.

In some embodiments, the recording is performed within 30 minutes after a meal. In some embodiments, the recording is performed within 30 minutes after swallowing a pill. In some embodiments, the episode of dysphagia is difficulty with food or pill going down. In some embodiments, the episode of dysphagia is difficulty with food going down, and the patient was not able to finish the rest of the meal as planned.

In some embodiments, in order to help get the food or pill down, the patient
a. Took slow, calm breaths;
b. Changed position;
c. Swallowed repeatedly;
d. Drank some liquid;
e. Drank a lot of liquid;
f. Coughed;
g. Made the food or pill come back up;
h. Went to the emergency room; or
i. Did not do anything to get the food or pill down.

In some embodiments, the present disclosure provides a non-transitory computer readable storage media device encoded with a computer program including instructions executable by a digital processing device for treating dysphagia in a patient in need thereof, comprising
(a) instructions configured to provide a questionnaire to a patient, wherein the questionnaire comprises: at least one input to record episode-based dysphagia events;
wherein said input records:
   (i) at least one question determining the severity of the dysphagia event;
   (ii) at least one question determining the pain associated with the dysphagia event; and
   (iii) at least one question determining the discomfort associated with the dysphagia event; and
(b) instructions configured to apply via the digital processing device an algorithm to answers to said questions to determine a score calculated over 1-21 days,
wherein the algorithm comprises:
   scoring the at least one severity question from 0 to 10
   scoring the at least one pain question from 0 to 10;
   scoring the at least one discomfort question from 0 to 10;
   summing the scores of all the questions presented in the questionnaire; and
   calculating the daily average score;
(c) evaluating the evaluating the daily score against a treatment range; and
(d) if the daily average score falls within the treatment range, the device is configured to instruct the administration of a therapeutic agent to the patient.

In some embodiments, the device further comprises: at least one input to record dysphagia events over 24-hours; wherein said input records:
(i) the approximate time of the dysphagia event;
(ii) at least one question determining the severity of each recorded dysphagia event;
   (iii) at least one question determining the pain associated with the dysphagia event; and
(iv) at least one question determining the discomfort associated with the dysphagia event.

In some embodiments, the device further comprises: instructions configured to apply via the digital processing device an algorithm to answers to said questions to determine a score calculated over 1-21 days,

wherein the algorithm comprises:
scoring the at least one severity question from 0 to 10;
scoring the at least one pain question from 0 to 10;
scoring the at least one discomfort question from 0 to 10;
summing the scores of all the questions presented in the questionnaire; and
calculating the daily average score.

In some embodiments, the device further comprises one input to provide a summary of the past 24 hours comprising:
(i) at least one question determining the types of food consumed;
(ii) at least one question determining the worst severe dysphagia episode of the day;
   (iii) at least one question determining the worst pain associated with a dysphagia episode of the day; and
(iv) at least one question determining the worst discomfort associated with a dysphagia episode of the day.

In some embodiments, the device further comprises instructions configured to apply via the digital processing device an algorithm to answers to said questions to determine a score calculated over 1-21 days,
wherein the algorithm comprises:
   scoring the at least one severity question from 0 to 10;
   scoring the at least one pain question from 0 to 10;
   scoring the at least one discomfort question from 0 to 10;
   summing the scores of all the questions presented in the questionnaire; and
   calculating the daily average score.

In some embodiments, the device further sums the following events over a 24-hour period:
a) the number of episode-based dysphagia events;
b) the number of 24-hour recorded dysphagia events;
c) the total number of dysphagia events;
d) the total duration of dysphagia events for episode-based dysphagia events; and
e) the total imputed duration of dysphagia for 24-hour recorded dysphagia events.

In some embodiments, the device further determines over a 24-hour period:
a) the worst difficulty score recorded in an episode-based dysphagia event;
b) the worst pain score recorded in an episode-based dysphagia event;
c) the worst discomfort score recorded in an episode-based dysphagia event;
d) the worst composite symptom summary score in an episode-based dysphagia event;
e) the worst difficulty score recorded in an 24-hour record;
f) the worst pain score recorded in an 24-hour record;
g) the worst discomfort score recorded in an 24-hour record; and
h) the worst composite symptom summary score in a 24-hour record.

In some embodiments, the device further determines over a 24-hour period:
a) the worst difficulty score recorded in any episode during the period;
b) the worst pain score recorded in any episode during the period;
c) the worst discomfort score recorded in any episode during the period; and
d) the worst composite symptom summary score during the period.

In some embodiments, the scores are calculated over the 1-21-day period:
a) the average difficulty score recorded on all episode-based dysphagia events;
b) the average pain score recorded on all episode-based dysphagia events;
c) the average discomfort score recorded on all episode-based dysphagia events;
d) the average difficulty score recorded on all 24-hour recorded dysphagia events;
e) the average pain score recorded on all 24-hour recorded dysphagia events;
f) the average discomfort score recorded on all 24-hour recorded dysphagia events;
g) the average difficulty score recorded on all dysphagia events;
h) the average pain score recorded on all dysphagia events;
i) the average discomfort score recorded on all dysphagia events;
j) the average difficulty score recorded on all summary recorded dysphagia events;
k) the average pain score recorded on all summary recorded dysphagia events; and
l) the average discomfort score recorded on all summary recorded dysphagia events.

In some embodiments, the device further calculates
a) the number of food types consumed over the 14-day period; and
b) the number of dysphagia-free days over the 14-day period.

In some embodiments, the device input further records
(iv) at least one question determining the type of food or pill involved in the dysphagia event;
(v) at least one question determining avoidance of solid food or pill; and
(vi) at least one question determining if the dysphagia was involved with food whether the patient completed the meal.

In some embodiments, the dysphagia is associated with eosinophilic esophagitis (EoE).

In some embodiments, the score is calculated over 14 days.

In some embodiments, the therapeutic agent is a corticosteroid.

In some aspects the present disclosure provides a method for treating dysphagia in a patient in need thereof, comprising
(a) instructions configured to provide a questionnaire to a patient, wherein the questionnaire comprises: at least one input to record episode-based dysphagia events; wherein said input records:
(i) at least one question determining the severity of the dysphagia event;
(ii) at least one question determining the pain associated with the dysphagia event; and
(ii) at least one question determining the discomfort associated with the dysphagia event; and
(b) instructions configured to apply via the digital processing device an algorithm to answers to said questions to determine a score calculated over 1-21 days,
wherein the algorithm comprises:
   scoring the at least one severity question from 0 to 10;
   scoring the at least one pain question from 0 to 10;
   scoring the at least one discomfort question from 0 to 10;
   summing the scores of all the questions presented in the questionnaire; and
   calculating the daily average score;
(c) evaluating the evaluating the daily average score against a threshold; and
(d) if the daily average score exceeds a threshold, administering a corticosteroid to the patient.

In some embodiments, the method further comprises at least one input to record dysphagia events over 24-hours; wherein said input records:
(i) the approximate time of the dysphagia event;
(ii) at least one question determining the severity of each recorded dysphagia event;
(iii) at least one question determining the pain associated with the dysphagia event; and
(iv) at least one question determining the discomfort associated with the dysphagia event.

In some embodiments, the method further comprises instructions configured to apply via the digital processing device an algorithm to answers to said questions to determine a score calculated over 1-21 days,
wherein the algorithm comprises:
   scoring the at least one severity question from 0 to 10;
   scoring the at least one pain question from 0 to 10;
   scoring the at least one discomfort question from 0 to 10;
   summing the scores of all the questions presented in the questionnaire; and
   calculating the daily average score.

In some embodiments, the device further comprises one input to provide a summary of the past 24 hours comprising:
(i) at least one question determining the types of food consumed;
(ii) at least one question determining the worst severe dysphagia episode of the day;
(iii) at least one question determining the worst pain associated with a dysphagia episode of the day; and
(iv) at least one question determining the worst discomfort associated with a dysphagia episode of the day.

In some embodiments, the device further comprises instructions configured to apply via the digital processing device an algorithm to answers to said questions to determine a score calculated over 1-21 days,
wherein the algorithm comprises:
   scoring the at least one severity question from 0 to 10;
   scoring the at least one pain question from 0 to 10;
   scoring the at least one discomfort question from 0 to 10;
   summing the scores of all the questions presented in the questionnaire; and
   calculating the daily average score.

In some embodiments, the device further sums the following events over a 24-hour period:
a) the number of episode-based dysphagia events;
b) the number of 24-hour recorded dysphagia events;
c) the total number of dysphagia events;
d) the total duration of dysphagia events for episode-based dysphagia events; and
e) the total imputed duration of dysphagia for 24-hour recorded dysphagia events.

In some embodiments, the device further determines over a 24-hour period:
a) the worst difficulty score recorded in an episode-based dysphagia event;
b) the worst pain score recorded in an episode-based dysphagia event;
c) the worst discomfort score recorded in an episode-based dysphagia event;
d) the worst composite symptom summary score in an episode-based dysphagia event;
e) the worst difficulty score recorded in an 24-hour record;
f) the worst pain score recorded in an 24-hour record;
g) the worst discomfort score recorded in an 24-hour record; and
h) the worst composite symptom summary score in a 24-hour record.

In some embodiments, the device further determines over a 24-hour period:
a) the worst difficulty score recorded in any episode during the period;
b) the worst pain score recorded in any episode during the period;
c) the worst discomfort score recorded in any episode during the period; and
d) the worst composite symptom summary score during the period.

In some embodiments, the scores are calculated over the 1-21-day period:
a) the average difficulty score recorded on all episode-based dysphagia events;
b) the average pain score recorded on all episode-based dysphagia events;
c) the average discomfort score recorded on all episode-based dysphagia events;
d) the average difficulty score recorded on all 24-hour recorded dysphagia events;
e) the average pain score recorded on all 24-hour recorded dysphagia events;
f) the average discomfort score recorded on all 24-hour recorded dysphagia events;
g) the average difficulty score recorded on all dysphagia events;
h) the average pain score recorded on all dysphagia events;
i) the average discomfort score recorded on all dysphagia events;
j) the average difficulty score recorded on all summary recorded dysphagia events;
k) the average pain score recorded on all summary recorded dysphagia events; and
l) the average discomfort score recorded on all summary recorded dysphagia events.

In some embodiments, the device further calculates a) the number of food types consumed over the 14-day period; and b) the number of dysphagia-free days over the 14-day period.

In some embodiments, the device input further records
(iv) at least one question determining the type of food or pill involved in the dysphagia event;
(v) at least one question determining avoidance of solid food or pill; and
(vi) at least one question determining if the dysphagia was involved with food whether the patient completed the meal.

In some embodiments, the dysphagia is associated with eosinophilic esophagitis (EoE).

In some embodiments, the score is calculated over 14 days.

### BRIEF DESCRIPTION OF THE FIGURES

FIG. 1 shows a representative example of an episode-based diary capturing dysphagia events in real-time (RTE).

FIG. 2 shows a representative example of a 24-hour diary capturing dysphagia events at the end of the day (EOD).

FIG. 3 is a graph showing Mean Ratings: Valid Days measured using the PRO instrument of the disclosure. Valid day is defined as completing the EOD record.

FIG. 4 is a graph showing Total Number of episodes: Baseline Period (−14 through −1)

Patients with ≥8 Valid Days of Reporting measured using the PRO instrument of the disclosure. Reporting is defined as completing the EOD record; Note: includes one patient with no episodes

FIG. 5 is a graph showing Percentage of episodes/day on days with at least one event Patient-days: Baseline Period (−14 through −1) measured using the PRO instrument of the disclosure.

FIG. 6 is a graph showing Food types, pill usage, allergy, and triggers; Patient-days: Baseline Period (−14 through −1). measured using the PRO instrument of the disclosure. Food types, allergy, and triggers only captured at EoD.

FIG. 7 is a graph showing Mean Ratings: Valid Days measured using the PRO instrument of the disclosure. Valid day is defined as completing the EOD record.

FIG. 8 is a graph showing Worst Difficulty from All episodes: Valid Days measured using the PRO instrument of the disclosure. Valid day is defined as completing the EOD record.

FIG. 9 is a graph showing Average Difficulty from All episodes: Valid Days measured using the PRO instrument of the disclosure. Valid day is defined as completing the EOD record.

FIG. 10 is a graph showing Worst Pain from All episodes: Valid Days measured using the PRO instrument of the disclosure. Valid day is defined as completing the EOD record.

FIG. 11 is a graph showing Average Pain from All episodes: Valid Days measured using the PRO instrument of the disclosure. Valid day is defined as completing the EOD record.

FIG. 12 is a graph showing Worst Discomfort from All episodes: Valid Days measured using the PRO instrument of the disclosure. Valid day is defined as completing the EOD record.

FIG. 13 is a graph showing Average Discomfort from All episodes: Valid Days measured using the PRO instrument of the disclosure. Valid day is defined as completing the EOD record.

FIG. 14 is a graph showing Worst Summary Rating from All episodes: Valid Days measured using the PRO instrument of the disclosure. Valid day is defined as completing the EOD record.

FIG. 15 is a graph showing Average Summary Rating from All episodes: Valid Days measured using the PRO instrument of the disclosure. Valid day is defined as completing the EOD record.

FIG. 16 illustrates by the logic flow method for assessing and/or treating dysphagia, the method as described herein.

### DETAILED DESCRIPTION

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

It should be understood that singular forms such as “a,” “an,” and “the” are used throughout this application for convenience, however, except where context or an explicit statement indicates otherwise, the singular forms are intended to include the plural. All numerical ranges should be understood to include each and every numerical point within the numerical range, and should be interpreted as reciting each and every numerical point individually. The endpoints of all ranges directed to the same component or property are inclusive, and intended to be independently combinable.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

The term “drug”, “active” or “active pharmaceutical ingredient” as used herein includes a pharmaceutically acceptable and topically acting corticosteroid, pharmaceutically acceptable salts, esters, solvates (including hydrates), polymorphs, stereoisomers, and/or prodrugs, and mixtures thereof. The terms “salts” refers to the product formed by the reaction of a suitable inorganic or organic acid with the “free base” form of the drug. Suitable acids include those having sufficient acidity to form a stable salt, for example acids with low toxicity such as the salt approved for use in humans or animals. Non-limiting examples of acids that may be used to form salts of an orally active drug, include inorganic acids, e.g., HCl, H₃PO₄, H₂SO₄. Non-limiting examples of organic acids include alkyl sulfonic acids and propionic acid.

The terms “pharmaceutical composition” and “pharmaceutical dosage form,” are used interchangeably herein to refer to an oral dosage form (suspension, solution, powder, solid, etc.) which can be used to administer a corticosteroid. Non-limiting examples of dosage forms include an orally disintegrating composition, such as a tablet, a lyophilized matrix, a film, and a wafer, liquid composition, a gel, a slurry, a lozenge, a lollipop, sachet, an effervescent tablet, and the like.

The term “oral corticosteroid” and “corticosteroid” are used interchangeably to refer to a corticosteroid which is administered orally, e.g., in a pharmaceutical composition described herein.

The terms “orally disintegrating dosage form”, “orally disintegrating tablet”, “orally dispersing tablet”, or “ODT” refer to a solid dosage form/tablet of the present disclosure, which disintegrates rapidly in the oral cavity of a patient after administration, without chewing, to form a suspension comprising the corticosteroid. The rate of oral disintegration can vary, but is significantly faster than the rate of oral disintegration of conventional solid dosage forms or chewable solid dosage forms (i.e., tablets or capsules) which are intended to be swallowed immediately after administration.

As used herein, the terms “treating,” “treatment” and “treat” include (i) preventing a particular disease or disorder from occurring in a subject who may be predisposed to the disease or disorder but has not yet been diagnosed as having it; (ii) curing, treating, or inhibiting the disease, i.e., arresting its development; or (iii) ameliorating the disease by reducing or eliminating symptoms, conditions, and/or by causing regression of the disease. In some embodiments, “treating,” “treatment” and “treat” may include administering a therapeutically effective regimen as defined herein.

The term “about”, as used herein to refer to a numerical quantity, includes “exactly” plus or minus up to 10% of that referenced numeric indication. When the term “about” is used in reference to a range of values, the term “about” refers to both the minimum and maximum value of the range (e.g., “about 1-50 μm” means “about 1 μm to about 50 μm”). The term “intimately associated”, as used herein to describe the spatial relationship between two or more components of a composition refers to components that are intimately mixed, such as, for example, in mixtures, coatings and matrices.

Unless indicated otherwise, all percentages and ratios are calculated by weight. Unless indicated otherwise, all percentages and ratios are calculated based on the total composition.

The term “having no significant systemic glucocorticoid or mineralocorticoid activity”, as used herein refers to corticosteroid compositions which do not provide a generalized effect in the body through absorption into the circulation, but do provide local effects through topical contact with a diseased tissue. Examples include fluticasone, flunisolide, budesonide, circlesone, mometasone, tixocortol, and beclomethasone. Corticosteroids which have high systemic glucocorticoid potencies when administered orally include e.g., hydrocortisone, prednisone, prednisolone, methylprednisolone, dexamethasone, betamethasone, etc. or mineralocorticoid potencies (e.g., alsosterone). Corticosteroids which typically have systemic glucocorticoid or mineralocorticoid activity when administered orally can also be used in the diluted compositions of the present disclosure, wherein the systemic uptake of the corticosteroid is reduced or suppressed.

A “histologic responder” may be defined as a subject who achieves a histologic response of peak eosinophils/HPF number ≤6 (as primary determinant). HPF may be defined as a standard area of 0.237 square millimeters in a microscope with 40× lens and 22 mm ocular.

A “histologic non-responder” may be defined as a subject who does not have a histologic response (i.e., do not achieve a histologic response of peak eosinophils/HPF number ≤6).

Subjects who develop food impaction with or without esophageal dilatation anytime during a study may be considered “treatment failures”.

The terms “PRO assessment”, “RPO tool”, “PRO questionnaire”, “PRO assessment questionnaire” and “PROSE” are used interchangeably herein.

### Methods of Treatment and Monitoring Using the PRO Assessment (PROSE)

Dysphagia may be monitored, evaluated, measured, diagnosed, and/or treated using the PRO assessment described herein. For example, gastrointestinal inflammatory disorders, such as eosinophilic esophagitis (EoE), an allergic/immune condition where the subject suffers from inflammation and/or swelling of the esophagus, affect a patient's ability to swallow food and can consequently cause malnutrition and failure to thrive. Such disorders may be caused by eosinophils in the esophagus. Typically, eosinophils are not found in the esophagus, but in EoE these cells accumulate and produce swelling that reduces the interior diameter of the esophagus making swallowing and eating very difficult. Often patients experience episodes of food impaction where food becomes lodged in the patient's esophagus, which can require emergency treatment. Because of the difficulty swallowing, and fear of food impaction, many patients with EoE limit themselves to eating soft foods such as yogurt, soups, and smoothies. In severe cases of EoE patients receive parenteral nutrition (e.g. intravenous feeding), which can provide required sustenance but limits the patient's activities and can lead to increased infection at the site of the catheter.

EoE most commonly occurs in Caucasian males and can occur at any age, with the symptoms varying with age. Infants and toddlers suffering from EoE may refuse food, fail to thrive, or experience “reflux” and/or vomiting. Young children typically report heartburn/reflux, abdominal pain, vomiting, food avoidance, and/or poor growth. For adults, the hallmark symptom is dysphagia (trouble swallowing), and EoE is implicated in over 50% of food impactions. Adult patients less commonly exhibit heartburn or chest pain. Adults with EoE also exhibit altered eating behavior such as dietary modifications, slow eating, excessive chewing, and increased consumption of liquids with food.

While the causes of EoE are not known, many EoE patients have a family history of allergies, asthma, and/or symptoms of allergic disorders (e.g. asthma, allergic rhinitis, atopic dermatitis, and food allergy). Additionally, environmental allergens such as dust mites, animals, pollen, and molds may play a role in the development of EoE. Because of the link between EoE and allergies, especially food allergies, elimination of the allergen may help alleviate symptoms. However, these types of elimination can be difficult to achieve. Further, because the symptoms of EoE resemble other gastrointestinal disorders, patients may be misdiagnosed and mistreated. For example, many patients with EoE are treated with Proton Pump Inhibitors (PPI), which can treat some symptoms of EoE but not inflammation, and whose long-term use has been linked to dementia, making their use in EoE patients less desirable.

The distinguishing feature of EoE is dysphagia. While current patient- and doctor-initiated questionnaires are used to track symptom improvement, based on a review of the currently existing PRO assessments of dysphagia, none are ideal. Indeed, many were found to be inadequate to support a co-primary endpoint based on dysphagia-episodes. While many of the existing instruments on the surface appear to cover content that is important to patients, concerns exist with the methods to score and identify dysphagia episodes using the current instruments in clinical trials, at least until qualitative research has been considered to fully understand the patient experience. For example, when patients limit their intake of difficult-to-swallow foods it could have the effect of making swallowing “easier” (i.e., improvement in dysphagia scores/occurrences) due to food avoidance rather than due to true disease improvement, thereby making score changes or counts of episodes using those dysphagia questions potentially difficult to interpret as true treatment benefit. Thus, as patients' symptoms become worse, and they therefore begin to limit difficult-to-swallow foods, current PRO assessments of dysphagia might actually show an “improvement” with reduced number of episodes reported simply because eating has been limited.

For example, Meritage (U.S. Pat. No. 10,176,301; US Patent App. Pub. No. 2016/0078186) uses a questionnaire that requires a patient to recall, at the end of the day, the number and severity of each episode of dysphagia that occurred over the last 24 hours. Needless to say, such a tool is not accurate because it relies on a patient's recollection concerning events of dysphagia occurring up to 24 hours earlier. Studies show that overtime, the patients do not accurately report the number of dysphasic episodes and under report the severity (e.g., pain, discomfort, and/or difficulty) of the episodes. Thus, current means to track episodes of dysphagia are not adequate to diagnose patients or as means to initiate a treatment regimen. New methods of not only addressing the inflammation causing the symptoms, but also accurately diagnosing, monitoring symptoms, and treating patients are required. The present disclosure provides methods of diagnosing, treating, monitoring, and managing treatment of dysphagia and/or inflammation associated with a gastrointestinal inflammatory disorder, such as dysphagia associated with EoE.

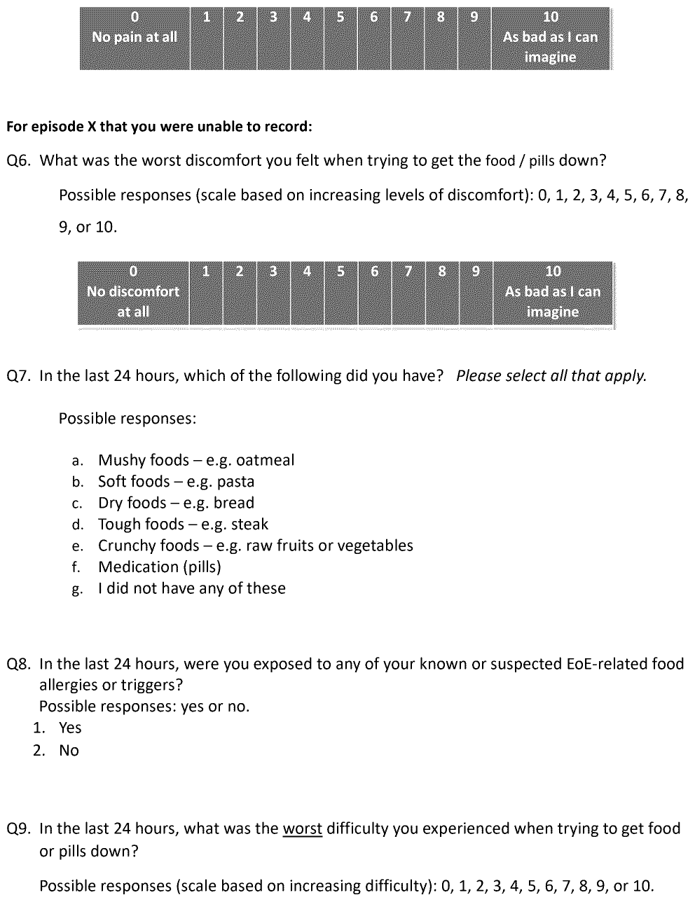

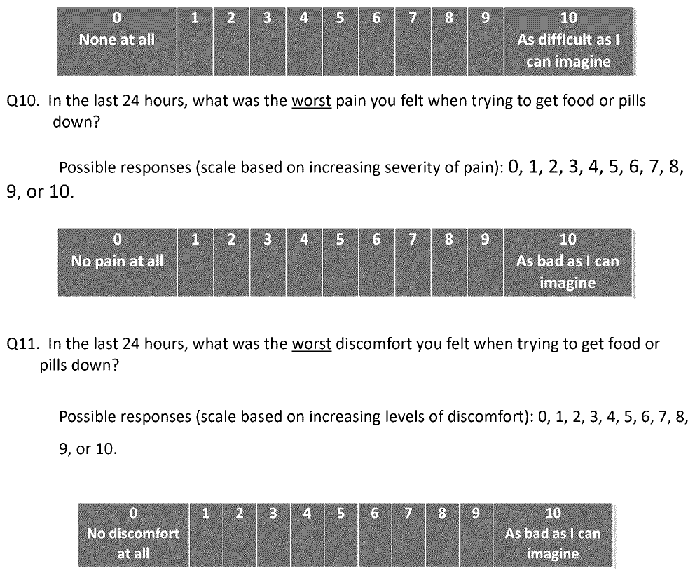

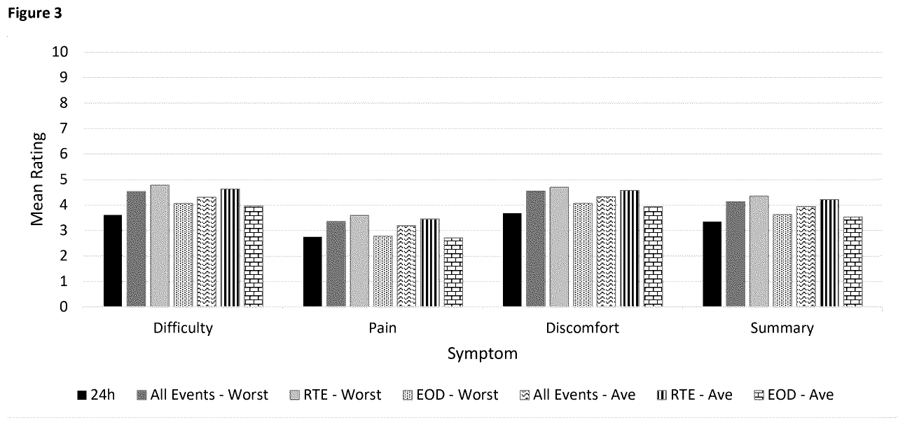

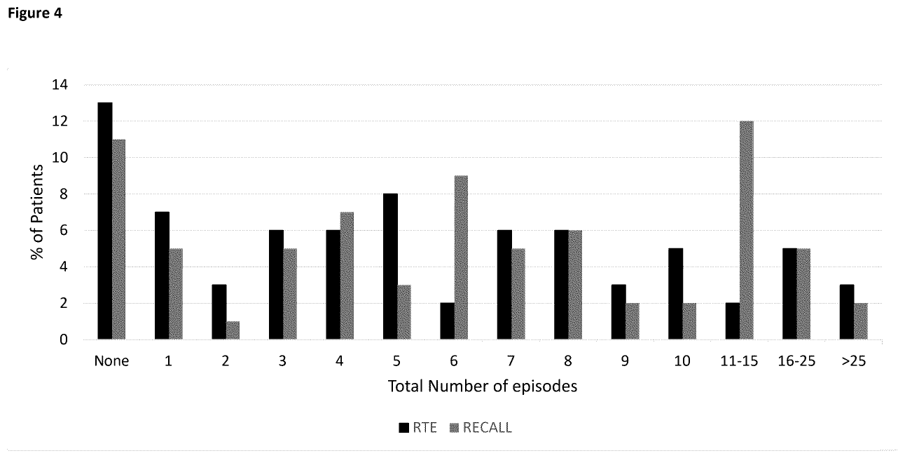

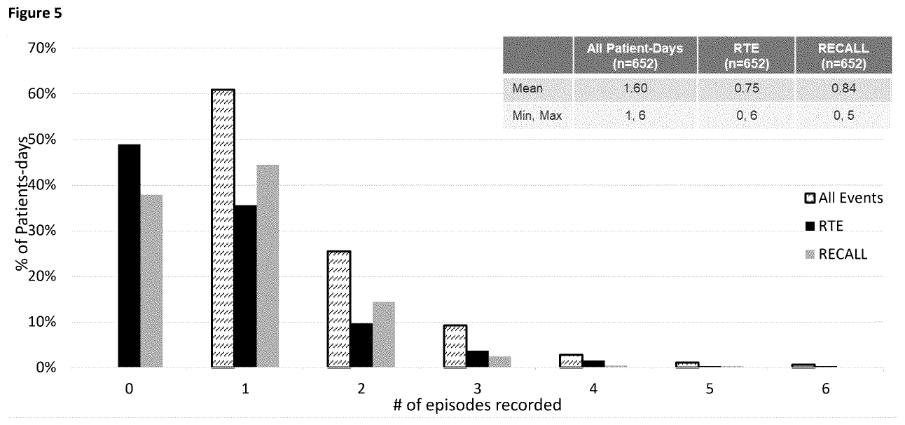

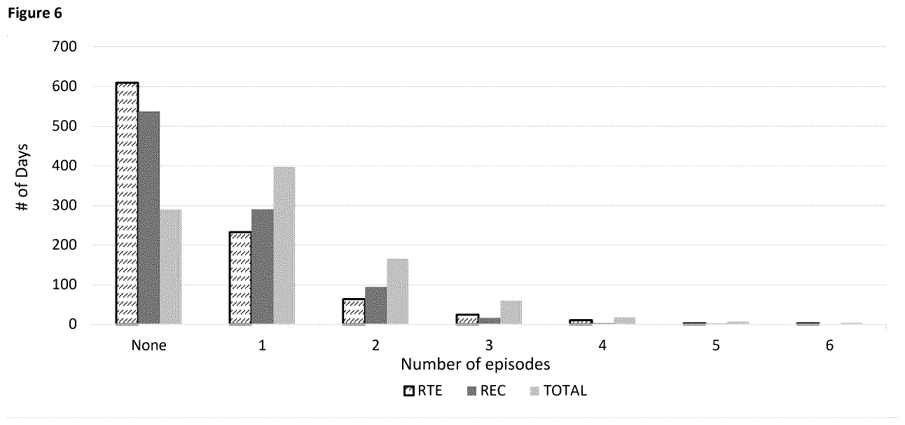

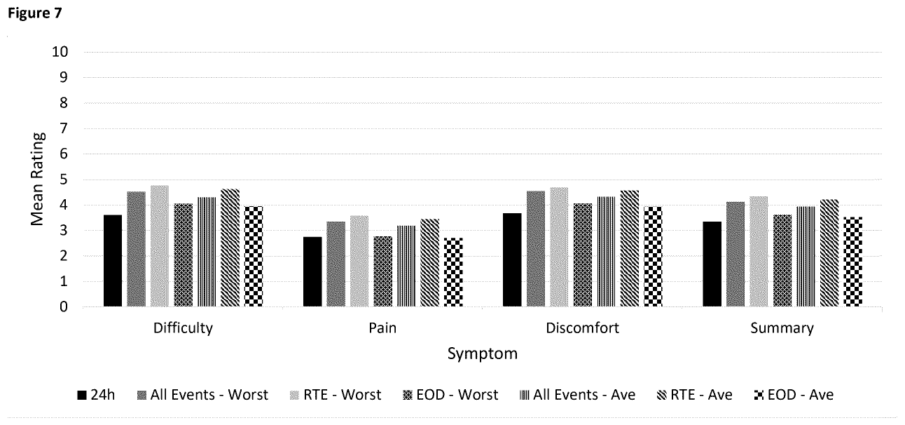

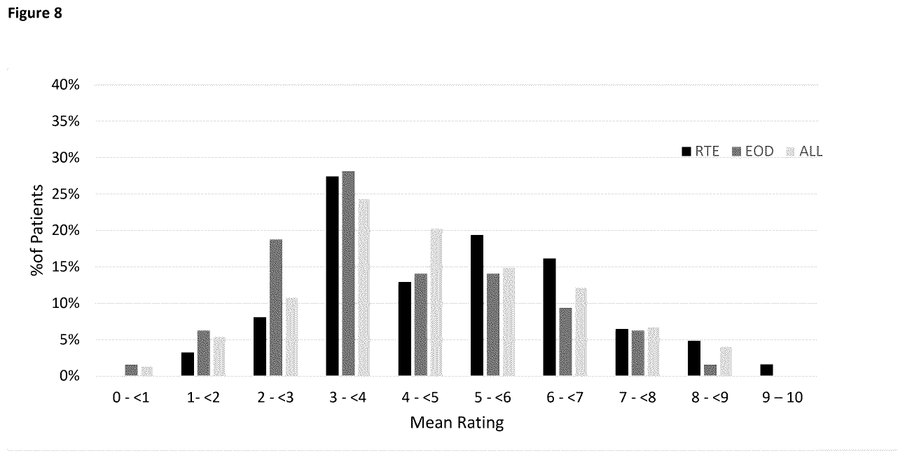

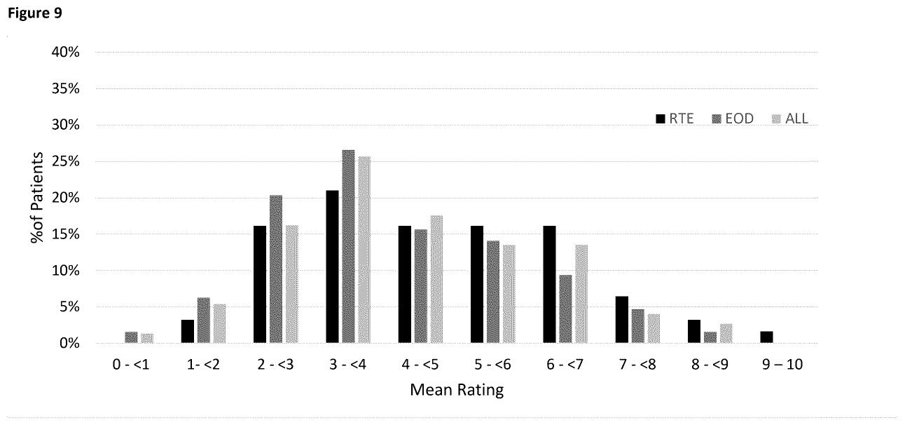

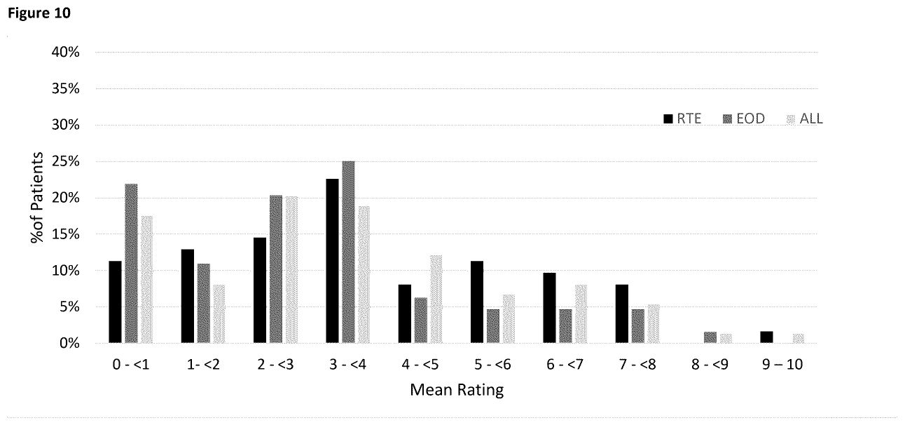

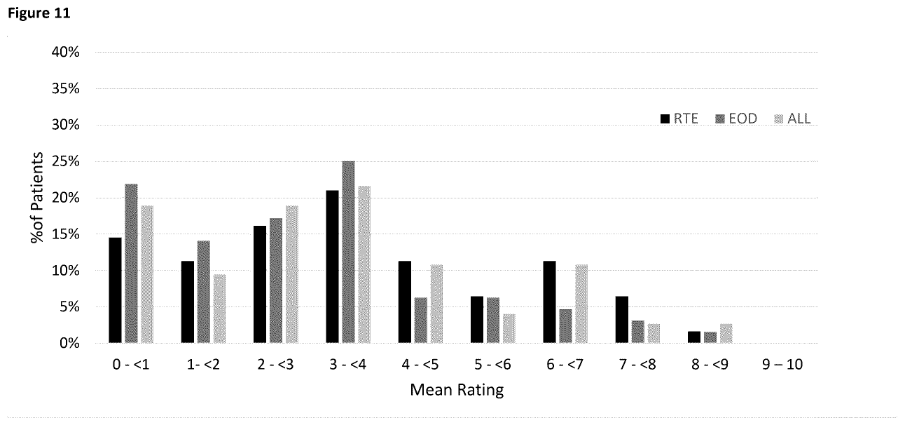

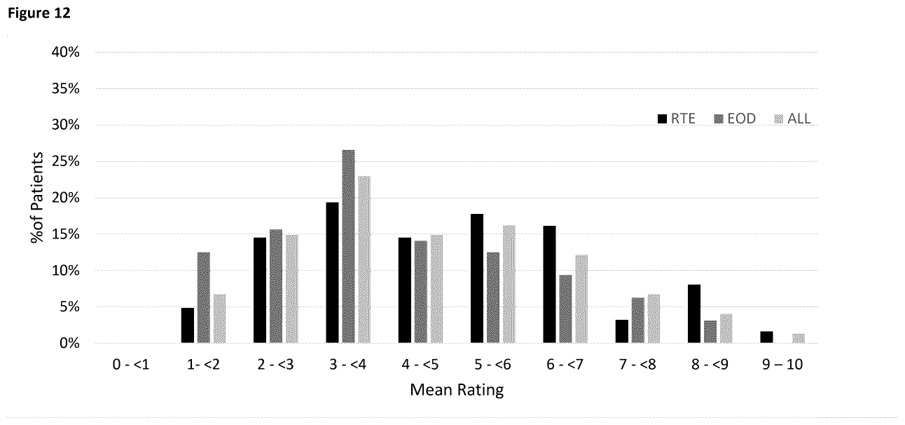

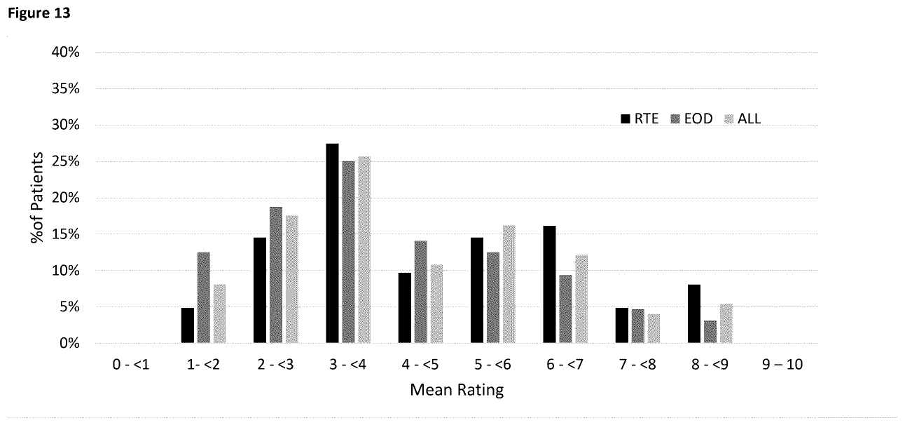

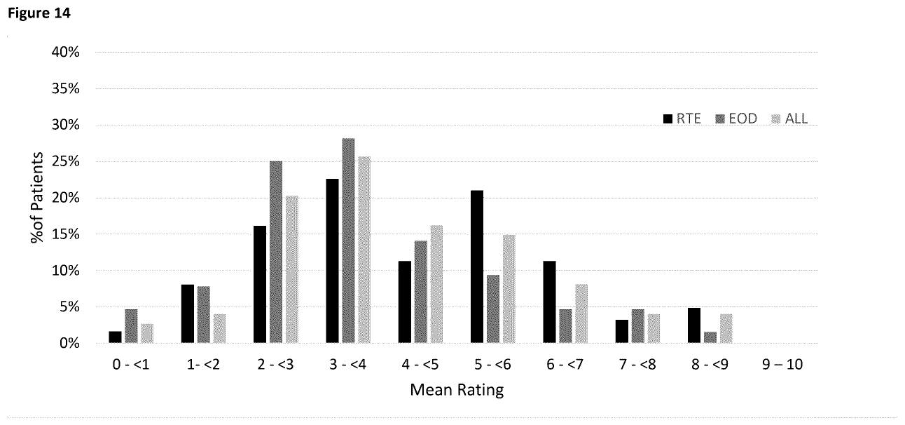

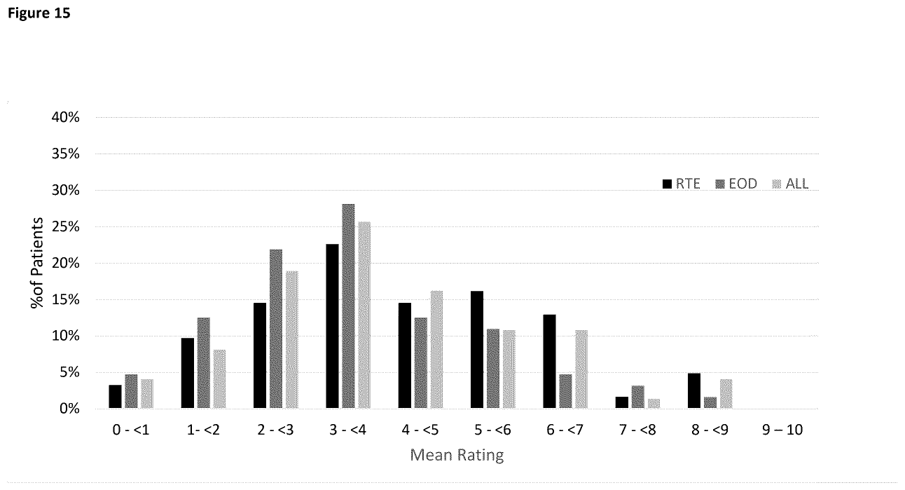

When patients enter information concerning the episode of dysphagia in real time (e.g., within 1 hour, within about 30 minutes, within about 15 minutes, or within about 10 minutes) in an episode-based diary in the PRO assessment disclosed herein, the severity (e.g., pain, discomfort, and/or difficulty) and number of episodes are more accurately recorded. This is shown in FIGS. 3-15. More specifically, patients recording episodes of dysphagia in real time report more events of dysphagia and a higher difficulty and discomfort score, compared to patients who recalled dysphagia episodes at the end of the day. Without being bound by theory, the lower pain score observed in real time recordation may be attributed inaccurate recollection of pain occurring earlier in the day. The PRO assessment thus allows for the selection of a particular patient population that can be treated for, inter alia, EoE with the therapeutic agents described herein. In some embodiments, only patients which have a specific mean score (i.e., a mean score falling within treatment range, as defined herein), as determined via the PRO questionnaire, are identified as suitable for treatment. In some embodiments, the treatment range is from about 2 to about 7 (e.g., 2, 3, 4, 5, 6, and 7). Patients with, inter alia, a score that falls within the treatment range are effectively treated with a therapeutic agent described herein (e.g., a corticosteroid) and show a meaningful reduction in the mean score determined via PRO questionnaire.

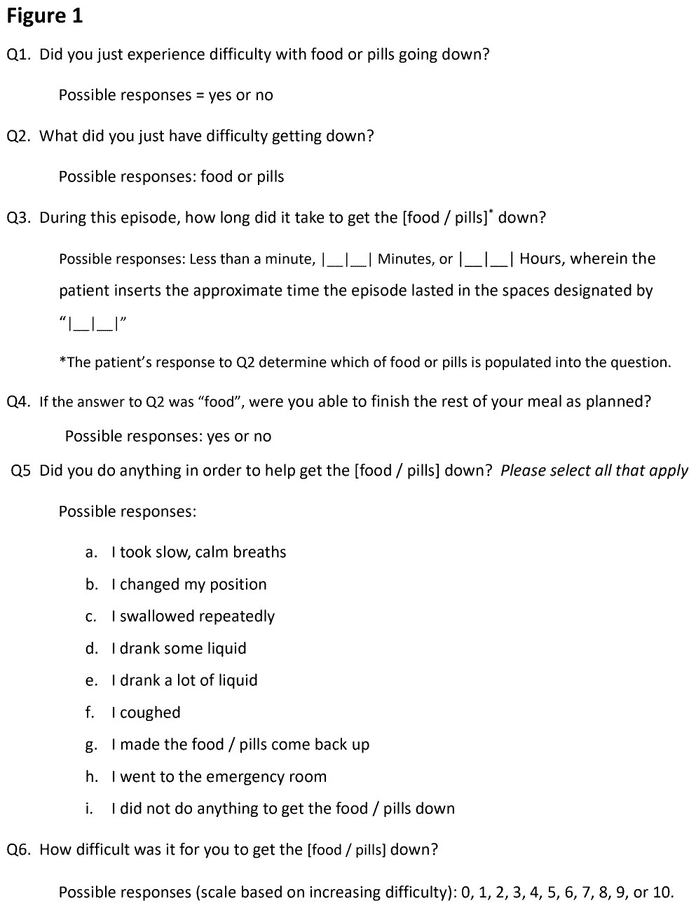

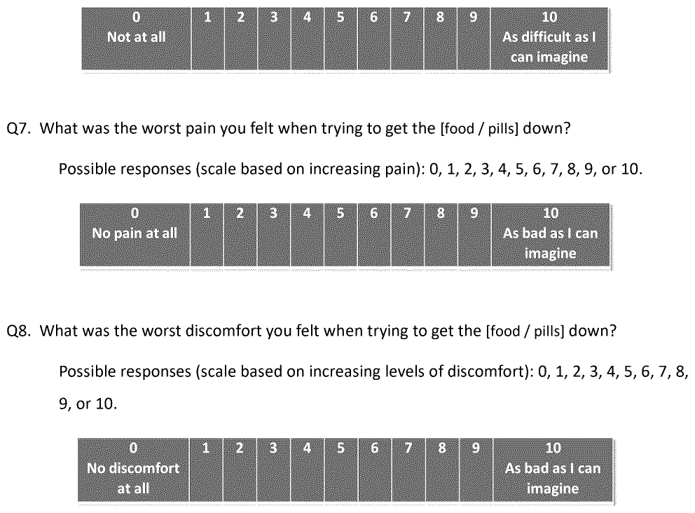

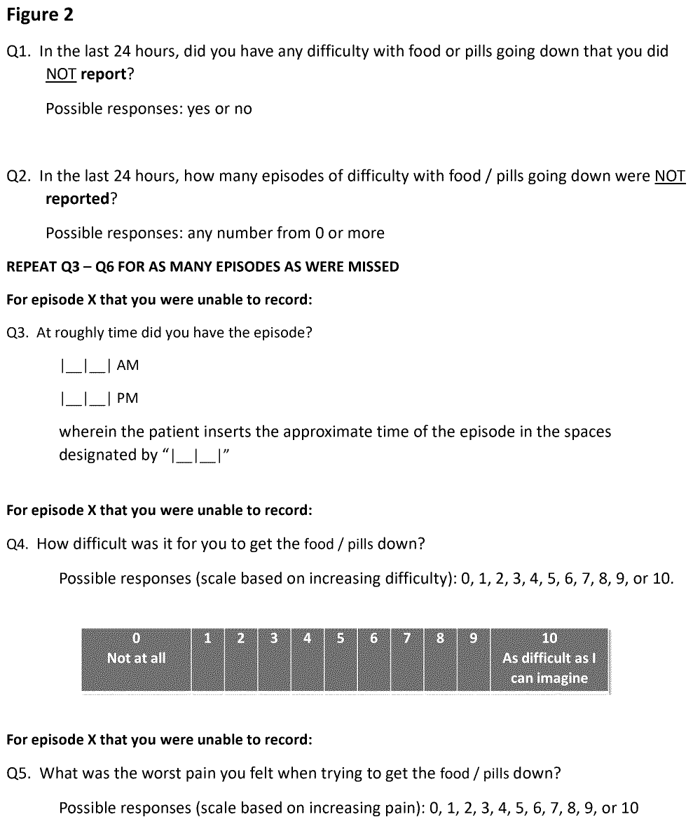

In some embodiments, the PRO assessment used in the methods disclosed herein improves on current assessments by providing a real-time assessment of patient symptoms by including an episode-based diary (FIG. 1). In some embodiments, the PRO assessment used in the methods disclosed herein further includes an end-of day catch diary ((e.g., FIG. 2) to record episodes that were missed during the day. In some embodiments, the PRO assessment used in the method disclosed herein further includes a 24-hour diary (e.g., US 2016/0078186, which is incorporated by reference in its entirety for all purposes). The PRO assessment disclosed herein provides a more accurate and sensitive assessment of patient dysphagia than those known in the art which only use recall-based entry (e.g. at the end of the day or week). “Episode-based diary” (or RTE) as used herein refers to a device used for recording various events associated with dysphagia (e.g., associated with EoE) in real time as such events occur, including, inter alia, (i) the severity, intensity, duration, pain, discomfort, difficulty, and/or frequency of dysphagia, (ii) type (including dosage form and active agent) and timing of treatment, and (iii) avoidance measures. The episode-based diary records and evaluates the events in order to assess the severity of the inflammation, diagnose the disease, and manage treatment of the disease. In some embodiments, the event is recorded within 1 hour, within 45 minutes, within 30 minutes, within 25 minutes, within 20 minutes, within 15 minutes, within 10 minutes, 9 minutes, within 8 minutes, within 7 minutes, within 6 minutes, within 5 minutes, within 4 minutes, within 3 minutes, within 2 minutes, or within 1 minute of the dysphagia occurrence of the event, inclusive of all values and ranges in between. “24-hour diary” as used herein refers to a device used for recording various events associated with dysphagia (e.g., associated with EoE) at the end of a 24 hour period—i.e., once a day, the patient recalls all of events associated with dysphagia that occurred over the previous 24 hour period, including, inter alia, (i) the severity, intensity, duration, pain, discomfort, difficulty, and/or frequency of dysphagia, (ii) type (including dosage form and active agent) and timing of treatment, and (iii) avoidance measures. In some embodiments, the patient records entries in the 24-hour diary after the last meal. In some embodiments, the patient records entries in the 24-hour diary about 6 pm, about 6:30 pm, about 7 pm, about 7:30 pm, about 8 pm, about 8:30 pm, about 9 pm, about 9:30 pm, about 10 pm, about 10:30 pm, about 11 pm, about 11:30 pm, or about 12 pm. The PRO assessment may also include an end of the day summary recording various aspects of the patient's dysphagic episodes during the day, including, inter alia, 1) the types of food eaten; 2) the presence of any allergy or triggers; 3) the worst dysphagic difficulty of the day; 4) the worst dysphagic pain of the day; and 5) the worst dysphagic discomfort of the day. This summary recording may be recorded at the same time as the patient records entries in the 24-hour diary or afterward.

As discussed herein, in some embodiments, the end-of-day diary may be used in combination with the episode-based diary, e.g., to capture episodes of dysphagia that the patient forgot to report and determine the mean daily score (as described herein). In some embodiments, the 24-hour diary may be used as an alternative, e.g., to record episodes of dysphagia while the patient is not be treated. Non-liming examples of using the 24-hour diary for the methods disclosed herein include diagnostic purposes before treatment, monitoring symptoms while the patient is off-treatment, such as when the patient is on a drug holiday or the inflammation or dysphagia has subsided. In other embodiments, the 24-hour diary may be used in combination with the episode-based diary.

Non-limiting examples of questions or events recorded in the episode-based diary include the following. In some embodiments, the patient reports whether they just experienced difficulty ingesting food or a pill (e.g., difficulty with food or pills going down). In some embodiments, the patient reports how long did took to get the food/pills down? Such an event may take a few seconds to several hours, e.g., from about 5 seconds to about 2 hours. If the patient had difficulty swallowing food, in some embodiments, the patient may report whether they were able to finish the meal. In some embodiments, the patient reports whether they did anything in order to help get the food/pills down, e.g., breathing slowly, adjusting physical position, swallowing repeatedly, coughing, regurgitating food/pills, drinking liquid, visiting doctor. The patient may record the measures taken to aid in getting the food/pill down, or the patient may select a measure from a list. Non-limiting examples of measures include: (a) “I took slow, calm breaths”; (b) “I changed my position”; (c) “I swallowed repeatedly”; (d) “I drank some liquid”; (e) “I drank a lot of liquid”; (f) “I coughed”; (g) “I made the food/pills come back up”; (h) “I went to the emergency room”; (i) “I did not do anything to get the food/pills down;” and (j) combinations thereof. In some embodiments, the patient reports how difficult was it for you to get the food/pills down. The difficulty can be reported using a scale of increasing severity from 0-10 (including 0, 1, 2, 3, 4, 5, 6, 7, 8, 9, and 10), with) being not difficult and 10 being as difficult as possible. In some embodiments, the patient reports the severity of the pain while trying to get the food/pills down. The severity can be reported using a scale of increasing severity from 0-10 (including 0, 1, 2, 3, 4, 5, 6, 7, 8, 9, and 10), where 0 is no pain and 10 is as bad as can be imaged. In some embodiments, the patient reports the severity of the discomfort while trying to get the food/pills down. The severity can be reported using a scale of increasing severity from 0-10 (including 0, 1, 2, 3, 4, 5, 6, 7, 8, 9, and 10), where 0 is no pain and 10 is as bad as can be imaged.

In some embodiments, the 24-hour diary is described in US 2016/0078186, which is herein incorporated by reference in its entirety. In some embodiments, the 24-hour diary is used in combination with the episode-based diary. In some embodiments, the 24-hour diary is used prior to starting treatment or while the patient is on a holiday from treatment (as described herein). Non-limiting examples of uses for 24-hour treatment include diagnosis, monitoring the number of days the patient is off treatment, notifying the patient when treatment should be reinitiated.

In some embodiments, the end of day diary is described in FIG. 2. Non-limiting examples of questions or events recorded in the episode-based diary include the following. In some embodiments, the patient reports if they have had any difficulty swallowing food or pills (e.g., if they have had difficulty with food or pills going down) over the last 24 hours. In some embodiments, the patient records the number of times they had difficulty swallowing food or pills over the last 24 hours. In some embodiments, the patient reports how many times they difficulty with food/pills going down that was not reported in the episode-based diary. In some embodiments, the patient reports the approximate time at which each episode of dysphagia occurred. In some embodiments, for each event of dysphagia, the patient reports how difficult it was to get the food/pills down. The difficulty can be reported using a scale of increasing severity from 0-10 (including 0, 1, 2, 3, 4, 5, 6, 7, 8, 9, and 10), where 0 is no pain and 10 is as difficult as can be imaged. In some embodiments, for each event of dysphagia, the patient reports the severity of the pain while trying to get the food/pills down. The severity can be reported using a scale of increasing severity from 0-10 (including 0, 1, 2, 3, 4, 5, 6, 7, 8, 9, and 10), where 0 is no pain and 10 is as bad as can be imaged. In some embodiments, for each event of dysphagia, the patient reports the severity of the discomfort while trying to get the food/pills down. The severity can be reported using a scale of increasing severity from 0-10 (including 0, 1, 2, 3, 4, 5, 6, 7, 8, 9, and 10), where 0 is no pain and 10 is as bad as can be imaged. In some embodiments, the patient report the types and/or identity of items that patient ingested over the previous 24 hour period, or the patient may select from a list the types and/or identity of items ingested. Non-limiting examples of such items include: (a) mushy foods—e.g. oatmeal; (b) soft foods—e.g. pasta; (c) dry foods—e.g. bread; (d) tough foods—e.g. steak; (e) crunchy foods—e.g. raw fruits or vegetables; (f) medication (pills); (g) I did not have any of these. The answers to these questions may help identify allergens or items that cause dysphagia. In some embodiments, the patient reports if, during the last 24 hours, they were exposed to any known or suspected EoE-related food allergies or triggers. In some embodiments, the patient reports the worst difficulty experienced over the last 24 hours in getting the food/pills down. The difficulty can be reported using a scale of increasing severity from 0-10 (including 0, 1, 2, 3, 4, 5, 6, 7, 8, 9, and 10), where 0 is no pain and 10 is as difficult as can be imaged. In some embodiments, the patient reports the worst pain felt over the last 24 hours while trying to get the food/pills down. The severity can be reported using a scale of increasing severity from 0-10 (including 0, 1, 2, 3, 4, 5, 6, 7, 8, 9, and 10), where 0 is no pain and 10 is as bad as can be imaged. In some embodiments, the patient reports the worst discomfort experienced over the last 24 hours while trying to get the food/pills down. The severity can be reported using a scale of increasing severity from 0-10 (including 0, 1, 2, 3, 4, 5, 6, 7, 8, 9, and 10), where 0 is no pain and 10 is as bad as can be imaged.

In some embodiments, the end-of-day diary is used in combination with the episode-based diary. In some embodiments, the end-of-day diary is used prior to starting treatment or while the patient is on a holiday from treatment (as described herein). Non-limiting examples of uses for end-of-day treatment include diagnosis, monitoring the number of days the patient is off treatment, notifying the patient when treatment should be reinitiated. In some embodiments, the end-of-day based diary is used to report events that the patients forgot to record in the episode based diary.

As discussed herein, in some embodiments, the method disclosed herein uses the PRO in combination with one or more methods to diagnose or treat dysphagia. Non-limiting examples of such diagnosis and treatment methods are disclosed herein.

In some embodiments, the PRO assessment is used for monitoring the effect of various treatments for dysphagia, including but not limited to, diet changes, proton pump inhibitors (PPI), dilating strictures, or therapeutic agents (e.g. corticosteroids, such as fluticasone propionate or biologics). In some embodiments, the PRO assessment is used to monitor the treatment of dysphagia in EoE patients, and measures the change from baseline in dysphagia episodes (a reduction in the number, severity, discomfort, and/or difficulty of dysphagia events by treatment arm compared to prior to treatment) as a co-primary endpoint. Moreover, this assessment measures episodes of behavior modification (e.g. food avoidance, increasing liquid intake, etc.) that may mask reports of actual dysphagia as measured by other assessments. For example, the results of the specific PRO assessment shown FIGS. 1-2 may be used as a co-endpoint with histology scores to determine if a patient is responding to treatment or not. The method can be used prior to treatment. Non-limiting examples of uses prior to treatment include diagnosis, notifying the patient when it is time to administer a drug. In some embodiments, the method is used to determine the type of treatment the patient presenting with dysphagia is administered.

### Drug Holiday

Many drugs are known to have side effects from continuous use. For example, continuous use of topical corticosteroids is known to cause: elevated pressure in the eyes (glaucoma); fluid retention, causing swelling in your lower legs; high blood pressure; problems with mood swings, memory and behavior and other psychological effects, such as confusion or delirium; weight gain, with fat deposits in your abdomen, face and the back of your neck; immune suppression; and growth retardation. In some embodiments, the methods described herein provide for a “drug holiday”—i.e., a period of time when the patient stops administering the drug in order to reduce side effects associated with continuous use—which improves the health and safety of the patient. Advantageously, after the drug holiday, the patient may resume treatment and the risk side effects is reduced relative to the risk if the patient had continuously used the drug. The drug holiday may last for any appropriate amount of time ranging from a few days up to a few years. In some embodiments, the drug holiday lasts for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 1.5 months, about 2 months, about 2.5 months, about 3 months, about 3.5 months, about 4 months, about 4.5 months, about 5 months, about 5.5 months, about 6 months, about 6.5 months, about 7 months, about 7.5 months, about 8 months, about 8.5 months, about 9 months, about 9.5 months, about 10 months, 10.5 months, about 11 months, about 11.5 months, about 1 year, about 1.5 years, about 2 years, about 2.5 years, about 3 years, about 3.5 years, about 4 years, about 4.5 years, about 5 years, or more, inclusive of all values and ranges therebetween. In some embodiments, after the drug holiday, the patient begins administering the drug.

In some embodiments, the patient reports (e.g., on a device and/or using a PRO assessment described herein) each time the patient administers the drug to treat dysphagia (e.g. associated with inflammation). In some embodiments, the device and/or the PRO assessment tracks the number of days (the duration of time) in which the drug was used. In some embodiments, after the patient has administered the drug for a particular amount of time, the patient is notified (via the device and/or the PRO assessment) to stop treatment.

In some embodiments, the patient reports (e.g., on a device and/or using a PRO assessment described herein) the date and identity of a side effect. In some embodiments, after the patient has reported the incidence of a particular side effect, or after the side effect has been reported an appropriate number of times (e.g., 1, 2, 3, 4, 5, 6, 7, 8, 9, 10 times, or more), the patient is notified (via the device and/or the PRO assessment) to stop treatment.

In some embodiments, the patient reports (e.g., on a device and/or using a PRO assessment described herein) each day in which the drug is not administered. In some embodiments, the device and/or the PRO assessment tracks the number of days (the duration of time) in which the drug was not used. In some embodiments, the patient also reports dysphagia episodes and other events associated with gastrointestinal inflammation including, inter alia, (i) the severity, intensity, duration, and/or frequency of dysphagia, and (ii) avoidance measures. In some embodiments, the patient uses the episode-based diary (e.g., as described herein or in FIG. 1), the 24 hour diary (e.g., as described herein or in FIG. 2), or combinations thereof.

### Gastrointestinal Inflammation and Methods of Treating and Monitoring the Same

In some embodiments, the present disclosure provides methods of monitoring or treating the symptoms associated with an inflammatory disorder of the intestinal tract. In some embodiments, the present disclosure provides methods of monitoring or treating inflammation associated with an inflammatory gastrointestinal disorder. In some embodiments the present disclosure provides methods of monitoring or treating both symptoms and inflammation associated an inflammatory gastrointestinal disorder. In some embodiments, the inflammatory gastrointestinal disorder affects the upper gastrointestinal tract. In some embodiments, the upper gastrointestinal tract is the esophagus.

Inflammatory gastrointestinal disorders which may be monitored or treated according to the present disclosure include, but are not limited to, inflammation of the esophagus, inflammation of the glottis, inflammation of the epiglottis, inflammation of the tonsils, inflammation of the oropharynx, eosinophilic esophagitis (EoE), gastroesophageal reflux disease (GERD), non-erosive reflux disease (NERD), erosive esophagitis, Barrett's esophagus, eosinophilic gastroenteritis, hypereosinophilic syndrome, corrosive (caustic) chemical esophagitis, radiation-induced esophagitis, chemotherapy-induced esophagitis, transient drug-induced esophagitis (also known as medication esophagitis), persistent drug-induced esophagitis, Crohn's disease of the esophagus, and pseudomembranous esophagitis. In some embodiments, the present disclosure includes a method for monitoring or treating a food allergy with an identified allergen, e.g., “atopic IBS”, and “atopic bowel”. In some embodiments, the present disclosure includes a method for monitoring or treating a patient having one or more of the above gastrointestinal disorders, wherein the patient also has a lactose allergy and/or a starch allergy. In some embodiments, the inflammatory gastrointestinal disorder is eosinophilic esophagitis (EoE). In some embodiments, the present disclosure includes a method for monitoring or treating a patient EoE, wherein the patient also a lactose allergy and/or a starch allergy.

In some embodiments, the pharmaceutical compositions disclosed herein are administered until symptoms and/or inflammation associated with gastrointestinal inflammation are treated. In some embodiments, the pharmaceutical compositions disclosed herein continue to be administered after symptoms and/or inflammation associated with gastrointestinal inflammation are treated. In some embodiments, the symptom is dysphagia, episodes of food impaction, feelings of having a lump in one's throat, and/or increased eosinophil count in the esophagus.

In some embodiments, the therapeutic agents disclosed herein (e.g., oral corticosteroid) contact and/or is deposited in the upper part of the gastrointestinal tract. In some embodiments, the therapeutic agent contacts and/or is deposited in the esophagus. In some embodiments, the oral corticosteroid contacts and/or is deposited in the distal portion of the esophagus. In some embodiments, the pharmaceutical composition contacts and/or is deposited in the proximal portion of the esophagus. In some embodiments, the oral corticosteroid contacts and/or is deposited in a substantially equivalent amount in the distal and proximal portion of the esophagus.

In addition to the PRO assessment and the methods of using the same as described herein, the treatment of gastrointestinal inflammation may also be measured by any means known in the art. For example, tests used to evaluate patients with esophageal inflammation such as EoE include, but are not limited to, biopsies, evaluation of symptoms (e.g. through patient reported outcome (PRO) or physician questionnaire), quality of life measurements, determination of Dysphagia-Free-Days in a patient, endoscopy (e.g. EREFS), esophageal compliance and/or improvement in esophageal remodeling (e.g. using a suitable diagnostic test such as EndoFLIP (available from Crospon Inc.), evaluation of biomarkers, decrease in peak eosinophil count, decrease in food impaction, and/or histology.

In some embodiments, administration of the therapeutic agents disclosed herein reduces mean score in the PRO questionnaire disclosed herein. In some embodiments, the correlation of the PRO questionnaire score with improvement in histological measurements (e.g. eosinophil count) indicates the patient's response to treatment.

In other embodiments, the correlation between the PRO questionnaire score and the patient's histological measurements (e.g. eosinophil count) indicates that the patient needs to be treated for dysphagia. For example, in some embodiments, a patient whose daily score is within the “treatment range” of from about 2-7 may also have an eosinophil count that is greater than or equal to 15 HPF

In some embodiments, a patient establishes a baseline mean score in the PRO assessment questionnaire prior to treatment. As discussed above, the baseline mean score indicates which patients are suitable for treatment. That is, if the patient has a baseline mean score that falls within the “treatment range” of from about 2-7, the PRO questionnaire instructs said patient to begin treatment for dysphagia. As used herein “baseline mean score” or “baseline mean questionnaire score” refers to the mean score measured using the PRO assessment described herein in an untreated patient that has an inflammatory condition which causes dysphagia (e.g., EoE). In some embodiments, the baseline mean score is the average of the daily score for a particular question described herein(e.g., pain, discomfort, or severity). In some embodiments, baseline mean score is the average of the mean scores for 2 or more questions described herein (e.g., pain and discomfort and/or severity; pain and severity and/or discomfort; etc). In some embodiments, the baseline mean score is the average of the maximum daily score for 2 or more questions described herein (e.g., pain and discomfort and/or severity; pain and severity and/or discomfort; etc). In some embodiments, the baseline mean score is the average daily score of the pain, difficulty and/or discomfort scores over a period of time. In some embodiments, the baseline mean score is measured from 1 day to 2 weeks prior to treatment, including 1, 2, 3, 4, 5, 6, 7, 8, 9, 10, 11, 12, 13, and 14 days, including all ranges therein. In particular embodiments, the baseline mean score is measured over 2 weeks. In some embodiments the baseline mean score is in the range of from 1 to 1,000, e.g., 10, 20, 30, 40, 50, 60, 70, 80, 90, 100, 150, 200, 250, 300, 350, 400, 450, 500, 550, 600, 650, 700, 750, 800, 850, 900, 950, and 1,000, inclusive of all values and subranges therebetween. In particular embodiments, the baseline mean score ranges from 1 to 10, e.g., 1, 2, 3, 4, 5, 6, 7, 8, 9, or 10, inclusive of all ranges therebetween. In particular embodiments, the baseline mean score ranges from 2 to 7, e.g., 2, 3, 4, 5, 6, or 7.

In some embodiments, the mean questionnaire score is measured in a treated patient between week 1 and year 10. In some embodiments, administration of the therapeutic agents disclosed herein reduces the baseline mean questionnaire score at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the mean questionnaire score in an untreated patient or the same patient before treatment. In some embodiments, administration of the therapeutic agents disclosed herein reduces the baseline mean questionnaire score for about 1 week, about 1 month, about 2 months, about 3 months, about 4 months, about 5 months, about 6 months, about 1 year, about 2 years, about 5 years, or about 10 years or more compared with the baseline mean questionnaire score in an untreated patient or the same patient before treatment. In some embodiments, the baseline mean questionnaire score is reduced by about 1%, about 5%, about 10%, about 20%, about 30%, about 40%, about 50%, about 60%, about 70%, about 80%, about 90%, or about 100%. In some embodiments, administration of the therapeutic agents disclosed herein reduces the baseline mean questionnaire score by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the therapeutic agents disclosed herein reduces the baseline mean questionnaire score by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more. In some embodiments, administration of the therapeutic agents disclosed herein reduces the baseline mean questionnaire score at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, the reduction from baseline is determined using the average score, as measured via the PRO questionnaire, over a period of from 1-21 days, e.g., 1, 2, 3, 4, 5, 6, 7, 8, 9, 10, 11, 12, 13, 14, 15, 16, 17, 18, 19, 20, and 21 days, inclusive of all ranges in between. In some embodiments, the reduction from baseline is determined over a 14 day period while the patient is on treatment. In some embodiments, the reduction from baseline is determined at week 12. In some embodiments, the reduction from baseline is determined over a 14 day period. Immediately preceding week 12 (i.e., days 70-83).

In some embodiments, the episode-based diary mean pain, discomfort, and/or difficulty score is measured in a treated patient between week 1 and year 10. In some embodiments, administration of the therapeutic agents disclosed herein reduces the baseline mean pain, discomfort, and/or difficulty score at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the therapeutic agents disclosed herein reduces the baseline mean pain, discomfort, and/or difficulty score for about 1 week, about 1 month, about 2 months, about 3 months, about 4 months, about 5 months, about 6 months, about 1 year, about 2 years, about 5 years, or about 10 years or more compared with the score in an untreated patient or the same patient before treatment. In some embodiments, the episode-based diary mean pain, discomfort, and/or difficulty score is reduced by about 1%, about 5%, about 10%, about 20%, about 30%, about 40%, about 50%, about 60%, about 70%, about 80%, about 90%, or about 100% compared to the baseline. In some embodiments, administration of the therapeutic agents disclosed herein reduces the episode-based diary mean pain, discomfort, and/or difficulty score by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared to the baseline. In some embodiments, administration of the therapeutic agents disclosed herein reduces the episode-based diary mean pain, discomfort, and/or difficulty score by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared to the baseline.

In some embodiments, the end-of-day catch diary (e.g., FIG. 2) median pain, discomfort, and/or difficulty score is measured in a treated patient between week 1 and year 10. In some embodiments, administration of the therapeutic agents disclosed herein reduces the end-of-da diary median pain, discomfort, and/or difficulty score as measured at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared to the baseline. In some embodiments, administration of the therapeutic agents disclosed herein reduces the end-of-day diary median pain, discomfort, and/or difficulty score for about 1 week, about 1 month, about 2 months, about 3 months, about 4 months, about 5 months, about 6 months, about 1 year, about 2 years, about 5 years, or about 10 years or more compared to the baseline. In some embodiments, the end-of-day diary median pain, discomfort, and/or difficulty score is reduced by about 1%, about 5%, about 10%, about 20%, about 30%, about 40%, about 50%, about 60%, about 70%, about 80%, about 90%, or about 100% compared with the baseline. In some embodiments, administration of the therapeutic agents disclosed herein reduces the end-of-day diary median pain, discomfort, and/or difficulty score by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared to the baseline. In some embodiments, administration of the therapeutic agents disclosed herein reduces the end-of-day diary median pain, discomfort, and/or difficulty score by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared to the baseline.

In some embodiments, the 24 hour recall diary mean pain, discomfort, and/or difficulty score is measured in a treated patient between week 1 and year 10. In some embodiments, administration of the therapeutic agents disclosed herein reduces the 24 hour recall diary mean pain, discomfort, and/or difficulty score as measured at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared to baseline. In some embodiments, administration of the therapeutic agents disclosed herein reduces the 24 hour recall diary mean pain, discomfort, and/or difficulty score for about 1 week, about 1 month, about 2 months, about 3 months, about 4 months, about 5 months, about 6 months, about 1 year, about 2 years, about 5 years, or about 10 years or more compared to baseline. In some embodiments, the 24 hour recall diary mean pain, discomfort, and/or difficulty score is reduced by about 1%, about 5%, about 10%, about 20%, about 30%, about 40%, about 50%, about 60%, about 70%, about 80%, about 90%, or about 100% compared to baseline. In some embodiments, administration of the therapeutic agents disclosed herein reduces the 24 hour recall diary mean pain, discomfort, and/or difficulty score by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared to baseline. In some embodiments, administration of the therapeutic agents disclosed herein reduces the 24 hour recall diary mean pain, discomfort, and/or difficulty score by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared to the baseline.

In some embodiments, the average of any two or more of the episode-based mean, end-of-day diary mean, and 24 hour recall mean pain, discomfort, and/or difficulty score is measured in a treated patient between week 1 and year 10. In some embodiments, administration of the therapeutic agents disclosed herein reduces the end of day diary median pain, discomfort, and/or difficulty score measured at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared to the baseline. In some embodiments, administration of the therapeutic agents disclosed herein reduces the average mean pain, discomfort, and/or difficulty score for about 1 week, about 1 month, about 2 months, about 3 months, about 4 months, about 5 months, about 6 months, about 1 year, about 2 years, about 5 years, or about 10 years or more compared to the baseline. In some embodiments, the end of day diary median pain, discomfort, and/or difficulty score is reduced by about 1%, about 5%, about 10%, about 20%, about 30%, about 40%, about 50%, about 60%, about 70%, about 80%, about 90%, or about 100% compared to the baseline. In some embodiments, administration of the therapeutic agents disclosed herein reduces average mean pain, discomfort, and/or difficulty score by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared to the baseline. In some embodiments, administration of the therapeutic agents disclosed herein reduces average mean pain, discomfort, and/or difficulty score by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared to baseline.

In some embodiments, the number of dysphagia-free days is measured in a treated patient between week 1 and year 10. In some embodiments, administration of the therapeutic agents disclosed herein increases the number of dysphagia-free days measured at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared to baseline. In some embodiments, administration of the therapeutic agents disclosed herein increases the number of dysphagia-free days for about 1 week, about 1 month, about 2 months, about 3 months, about 4 months, about 5 months, about 6 months, about 1 year, about 2 years, about 5 years, or about 10 years or more compared to baseline. In some embodiments, the number of dysphagia-free days is increased by about 1%, about 5%, about 10%, about 20%, about 30%, about 40%, about 50%, about 60%, about 70%, about 80%, about 90%, or about 100% compared to baseline. In some embodiments, administration of the therapeutic agents disclosed herein increases the number of dysphagia-free days by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared to baseline. In some embodiments, administration of the therapeutic agents disclosed herein increases dysphagia-free days by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared with the number of days in an untreated patient or the same patient before treatment.

In some embodiments, the response to episode-based dairy Q1 (“Did you just experience difficulty with food or pills going down”) is “yes”. In some embodiments, the patient answers “yes” to Q1 each time the patient ingests food or pills. In some embodiments, the patient answers “yes” about 95%, about 90%, about 85%, about 80%, about 75%, about 70%, about 65%, about 60%, about 55%, about 50%, about 45%, about 40%, about 35%, or about 30% (inclusive of all values and ranges therebetween) of the time. In some embodiments, the number of times that patient answers “yes” is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges therebetween, after administration of the therapeutic agent.

In some embodiments, the response to episode-based dairy Q3 (“During this episode, how long did it take to get the [food/pills] down”) is in the range of from 5 second to about 20 minutes. In some embodiments, the time it took for the food/pills to go down is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges therebetween, after administration of the therapeutic agent. In some embodiments, the time it took for the food/pills to go down is reduced by about 1 seconds, 2 seconds, 3 seconds, 4 seconds, 5 seconds, 6 seconds, 7 seconds, 8 seconds, 9 seconds, 10 seconds, 15 seconds, 20 seconds, 25 seconds, 30 seconds, 35 seconds, 40 seconds, 45 seconds, 50 seconds, 60 seconds, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, inclusive of all values and ranges therebetween, after administration of the therapeutic agent.

In some embodiments, the response to episode-based dairy Q4 (“If the answer to Q2 was “food”, were you able to finish the rest of your meal as planned?”) is no. In some embodiments, In some embodiments, the patient answers “no” about 95%, about 90%, about 85%, about 80%, about 75%, about 70%, about 65%, about 60%, about 55%, about 50%, about 45%, about 40%, about 35%, or about 30% (inclusive of all values and ranges therebetween) of the time. In some embodiments, the number of times that patient answers “no” is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges therebetween, after administration of the therapeutic agent. In some embodiments, after the patient has been administered a therapeutic agent, the patient answers “yes” about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the time, inclusive of all values and ranges therebetween.

In some embodiments, in response to episode-based dairy Q5 (“Did you do anything in order to help get the [food/pills] down”) the patient indicates the remediation measured the patient adopted. Non-limiting examples of possible responses include: “I took slow, calm breaths”; “I changed my position”; “I swallowed repeatedly”; “I drank some liquid”; “I drank a lot of liquid”; “I coughed”; “I made the food/pills come back up”; “I went to the emergency room”; and “I did not do anything to get the food/pills down”.

In some embodiments, the response to daily diary Q6 (“How difficult was it for you to get the [food/pills] down”] is 1, 2, 3, 4, 5, 6, 7, 8, 9, or 10. In some embodiments, the patient response to daily diary Q6 is independently 1, 2, 3, 4, 5, 6, 7, 8, 9, or 10 each time the patient tries to get the food/pills down. In some embodiments, the patient's response to daily diary Q6 is 0, 1, 2, 3, 4, 5, 6, 7, or 8 after administration of the therapeutic agent. In some embodiments, the patient's response to daily diary Q6 is reduced by 1, 2, 3, 4, 5, 6, 7, 8, 9, or 10 after administration of the therapeutic agent. In some embodiments, the patient's sum total response to Q6 during treatment is reduced by about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% compared to the baseline response to Q6. As used herein “baseline response” is the daily mean score for a particular question prior to treatment as determined using the PRO questionnaire.

In some embodiments, the response to episode-based dairy Q7 (“What was the worst pain you felt when trying to get the [food/pills] down?”) is 1, 2, 3, 4, 5, 6, 7, 8, 9, or 10. In some embodiments, the patient response to daily diary Q7 is independently 1, 2, 3, 4, 5, 6, 7, 8, 9, or 10 each time the patient tries to get the food/pills down. In some embodiments, the patient's response to daily diary Q7 is 0, 1, 2, 3, 4, 5, 6, 7, or 8 after administration of the therapeutic agent. In some embodiments, the patient's response to daily diary Q7 is reduced by 1, 2, 3, 4, 5, 6, 7, 8, 9, or 10 after administration of the therapeutic agent. In some embodiments, the patient's sum total response to Q7 during treatment is reduced by about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% compared to the baseline response to Q7.

In some embodiments, the response to episode-based dairy Q8 (“What was the worst discomfort you felt when trying to get the [food/pills] down?”) is 1, 2, 3, 4, 5, 6, 7, 8, 9, or 10. In some embodiments, the patient response to daily diary Q8 is independently 1, 2, 3, 4, 5, 6, 7, 8, 9, or 10 each time the patient tries to get the food/pills down. In some embodiments, the patient's response to daily diary Q8 is 0, 1, 2, 3, 4, 5, 6, 7, or 8 after administration of the therapeutic agent. In some embodiments, the patient's response to daily diary Q8 is reduced by 1, 2, 3, 4, 5, 6, 7, 8, 9, or 10 after administration of the therapeutic agent. In some embodiments, the patient's sum total response to Q8 during treatment is reduced by about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% compared to the baseline response to Q8.

In some embodiments, the patient records episodes of dysphagia in the 24-hour diary.

In some embodiments, the response to 24-hour diary Q1 (“In the last 24 hours, did you have any difficulty with food or pills going down that you did NOT report?) is “yes”. In some embodiments, In some embodiments, the patient answers “yes” about 95%, about 90%, about 85%, about 80%, about 75%, about 70%, about 65%, about 60%, about 55%, about 50%, about 45%, about 40%, about 35%, or about 30% (inclusive of all values and ranges therebetween) of the time. In some embodiments, the number of times that patient answers “yes” is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and ranges therebetween, after administration of the therapeutic agent.

In some embodiments, the response to 24-hour diary Q2 (“In the last 24 hours, how many episodes of difficulty with food/pills going down were NOT reported?”) is 0, 1, 2, 3, 4, 5, 6, 7, 8, 9, 10, 11, 12, 13, 14, 15, 16, 17, 18, 19, or 20. In some embodiments, the patient response to 24-hour diary Q2 is reduced by about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% compared to the baseline response to Q2. In some embodiments, the patient response to 24-hour diary Q2 is reduced by about 1, 2, 3, 4, 5, 6, 7, 8, 9, 10, 11, 12, 13, 14, 15, 16, 17, 18, 19, 20 or more compared to the baseline response to Q2.

In some embodiments, the response the 24-hour diary Q3 (“At roughly time did you have the episode?”) is any time in the range of from 12 am to 12 pm, e.g., 12 am, 1 am, 2 am, 3 am, 4 am, 5 am, 6 am, 7 am, 8 am, 9 am, 10 am, 11 am, 12 pm, 1 pm, 2 pm, 3 pm, 4 pm, 5 pm, 6 pm, 7 pm, 8 pm, 9 pm, 10 pm, 11 pm, or 12 pm, including all time points therein. In some embodiments, the patient records a response to 24-hour diary Q3 for each instance of dysphagia.

In some embodiments, the response to 24-hour diary Q4 (“How difficult was it for you to get the food/pills down?”) is 1, 2, 3, 4, 5, 6, 7, 8, 9, or 10. In some embodiments, the patient response to daily diary Q4 is independently 1, 2, 3, 4, 5, 6, 7, 8, 9, or 10 each time the patient tried to get the food/pills down. In some embodiments, the patient's response to 24-hour diary Q4 is 0, 1, 2, 3, 4, 5, 6, 7, or 8 after administration of the therapeutic agent. In some embodiments, the patient's response to 24-hour diary Q4 is reduced by 1, 2, 3, 4, 5, 6, 7, 8, 9, or 10 after administration of the therapeutic agent. In some embodiments, the patient's sum total response to 24-hour diary Q4 during treatment is reduced by about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% compared to the baseline response to Q4.

In some embodiments, the response to 24-hour diary Q5 (“What was the worst pain you felt when trying to get the food/pills down?”) is 1, 2, 3, 4, 5, 6, 7, 8, 9, or 10. In some embodiments, the patient response to 24-hour diary Q5 is independently 1, 2, 3, 4, 5, 6, 7, 8, 9, or 10 each time the patient tries to get the food/pills down. In some embodiments, the patient's response to 24-hour diary Q5 is 0, 1, 2, 3, 4, 5, 6, 7, or 8 after administration of the therapeutic agent. In some embodiments, the patient's response to 24-hour diary Q5 is reduced by 1, 2, 3, 4, 5, 6, 7, 8, 9, or 10 after administration of the therapeutic agent. In some embodiments, the patient's sum total response to 24-hour diary Q5 during treatment is reduced by about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% compared to the baseline response to Q5.

In some embodiments, the response to 24-hour diary Q6 (“What was the worst discomfort you felt when trying to get the food/pills down?” is 1, 2, 3, 4, 5, 6, 7, 8, 9, or 10. In some embodiments, the patient response to 24-hour diary Q6 is independently 1, 2, 3, 4, 5, 6, 7, 8, 9, or 10 each time the patient tries to get the food/pills down. In some embodiments, the patient's response to 24-hour diary Q6 is 0, 1, 2, 3, 4, 5, 6, 7, or 8 after administration of the therapeutic agent. In some embodiments, the patient's response to 24-hour diary Q6 is reduced by 1, 2, 3, 4, 5, 6, 7, 8, 9, or 10 after administration of the therapeutic agent. In some embodiments, the patient's sum total response to 24-hour diary Q6 during treatment is reduced by about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% compared to the baseline response to Q6.

In some embodiments, in response to 24-hour diary Q7 (“In the last 24 hours, which of the following did you have?”) the patient indicates which avoidance measures the patient took. Non-limiting examples of avoidance measures include: Mushy foods—e.g. oatmeal; Soft foods—e.g. pasta; Dry foods—e.g. bread; Tough foods—e.g. steak; Crunchy foods—e.g. raw fruits or vegetables; Medication (pills); and I did not have any of these.

In some embodiments, the response to 24-hour diary Q9 (“In the last 24 hours, what was the worst difficulty you experienced when trying to get food or pills down?”) is 1, 2, 3, 4, 5, 6, 7, 8, 9, or 10. In some embodiments, the patient response to 24-hour diary Q9 is independently 1, 2, 3, 4, 5, 6, 7, 8, 9, or 10 each time the patient tries to get the food/pills down. In some embodiments, the patient's response to 24-hour diary Q9 is 0, 1, 2, 3, 4, 5, 6, 7, or 8 after administration of the therapeutic agent. In some embodiments, the patient's response to 24-hour diary Q6 is reduced by 1, 2, 3, 4, 5, 6, 7, 8, 9, or 10 after administration of the therapeutic agent. In some embodiments, the patient's sum total response to 24-hour diary Q9 during treatment is reduced by about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% compared to the baseline response to Q9.

In some embodiments, the response to 24-hour diary Q10 (“In the last 24 hours, what was the worst pain you felt when trying to get food or pills down?”) is 1, 2, 3, 4, 5, 6, 7, 8, 9, or 10. In some embodiments, the patient response to 24-hour diary Q10 is independently 1, 2, 3, 4, 5, 6, 7, 8, 9, or 10 each time the patient tries to get the food/pills down. In some embodiments, the patient's response to 24-hour diary Q10 is 0, 1, 2, 3, 4, 5, 6, 7, or 8 after administration of the therapeutic agent. In some embodiments, the patient's response to 24-hour diary Q10 is reduced by 1, 2, 3, 4, 5, 6, 7, 8, 9, or 10 after administration of the therapeutic agent. In some embodiments, the patient's sum total response to 24-hour diary Q10 during treatment is reduced by about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% compared to the baseline response to Q10.

In some embodiments, the response to 24-hour diary Q11 (“In the last 24 hours, what was the worst discomfort you felt when trying to get food or pills down?”) is 1, 2, 3, 4, 5, 6, 7, 8, 9, or 10. In some embodiments, the patient response to 24-hour diary Q11 is independently 1, 2, 3, 4, 5, 6, 7, 8, 9, or 10 each time the patient tries to get the food/pills down. In some embodiments, the patient's response to 24-hour diary Q11 is 0, 1, 2, 3, 4, 5, 6, 7, or 8 after administration of the therapeutic agent. In some embodiments, the patient's response to 24-hour diary Q11 is reduced by 1, 2, 3, 4, 5, 6, 7, 8, 9, or 10 after administration of the therapeutic agent. In some embodiments, the patient's sum total response to 24-hour diary Q11 during treatment is reduced by about 10%, about 15%, about 20%, about 25%, about 30%, about 40%, about 45%, about 50%, about 55%, about 60%, about 65%, about 70%, about 75%, about 80%, about 85%, about 90%, about 95%, or about 100% compared to the baseline response to Q11.

In some embodiments, patient response to treatment is determined by measuring changes in the score measured by the PRO assessment described herein (e.g., in FIG. 1 and/or in FIG. 2) in combination with a biological response such as histology score (e.g. eosinophil count).

In some embodiments, patient response is evaluated by assessing histology scores in a patient. In some embodiments the histology score is assessed by one or more different histologic features, including but not limited to, eosinophil inflammation, basal zone hyperplasia, dilated intercellular spaces, lamina propria fibrosis, eosinophil abscess, surface layering, surface epithelial alteration, and dyskeratotic epithelial cells.

In some embodiments, histology scores are measured prior to initiating treatment according to the present methods. In some embodiments, histology scores are measured at least two weeks after initiating treatment according to the present methods. In some embodiments, histology scores are measured prior to initiating treatment according to the present methods, and at least two weeks after initiating such treatment.

In some embodiments, administration of the therapeutic agent according to the methods disclosed herein reduces a histology score in a treated patient compared to an untreated patient or the same patient before treatment. In some embodiments, the histology score is measured in a treated patient between week 1 and year 10. In some embodiments, administration of the pharmaceutical compositions disclosed herein reduces a histology score at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the histology score in an untreated patient or the same patient before treatment. In some embodiments, administration of the pharmaceutical compositions disclosed herein reduces a histology score for about 1 week, about 1 month, about 2 months, about 3 months, about 4 months, about 5 months, about 6 months, about 1 year, about 2 years, about 5 years, or about 10 years, or more compared with the histology score in an untreated patient or the same patient before treatment.

In some embodiments, a histology score is reduced by about 1%, about 5%, about 10%, about 20%, about 30%, about 40%, about 50%, about 60%, about 70%, about 80%, about 90%, or about 100% compared with the histology score in an untreated patient or the same patient before treatment. In some embodiments, administration of the pharmaceutical compositions disclosed herein reduces the histology score by about 1%, about 5%, about 10%, about 20%, about 30%, about 40%, about 50%, about 60%, about 70%, about 80%, about 90%, or about 100% at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with the histology score in an untreated patient or the same patient before treatment. In some embodiments, administration of the pharmaceutical compositions disclosed herein reduces the histology score by about 1%, about 5%, about 10%, about 20%, about 30%, about 40%, about 50%, about 60%, about 70%, about 80%, about 90%, or about 100% for about 1 week, about 1 month, about 2 months, about 3 months, about 4 months, about 5 months, about 6 months, about 1 year, about 2 years, about 5 years, or about 10 years or more compared with the histology score in an untreated patient or the same patient before treatment.

In some embodiments, prior to treatment, the patient has a peak eosinophil that is greater than or equal to 15/HPF. In some embodiments, administration of the therapeutic agent disclosed herein reduces peak eosinophil in at least one biopsy to less than 15/HPF. In some embodiments, administration of the therapeutic according to the methods disclosed herein reduces peak eosinophil (per high power field (HPF) in at least one biopsy in a treated patient compared to peak eosinophil per HPF in an untreated patient or the same patient before treatment. In some embodiments, administration of the therapeutic agent disclosed herein reduces peak eosinophil in at least one biopsy to less than 15/HPF. In some embodiments, administration of the pharmaceutical compositions disclosed herein reduces peak eosinophil in at least one biopsy in a treated patient to less than about 14/HPF, less than about 13/HPF, less than about 12/HPF, less than about 11/HPF, less than about 10/HPF, less than about 9/HPF, less than about 8/HPF, less than about 7/HPF, less than about 6/HPF, less than about 5/HPF, less than about 4/HPF, less than about 3/HPF, less than about 2/HPF, less than about 1/HPF, or less (e.g. 0) in the patient. In some embodiments, administration of the therapeutic agent disclosed herein reduce peak eosinophil in at least one biopsy to less than 1 HPF in the patient. In some embodiments, the reduction of peak eosinophil in at least one biopsy in a treated patient is measured between about week 1 and about year 10. In some embodiments, the reduction of peak eosinophil is measured at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the therapeutic agent disclosed herein reduces peak eosinophil in at least one biopsy to less than about 14/HPF, less than about 13/HPF, less than about 12/HPF, less than about 11/HPF, less than about 10/HPF, less than about 9/HPF, less than about 8/HPF, less than about 7/HPF, less than about 6/HPF, less than about 5/HPF, less than about 4/HPF, less than about 3/HPF, less than about 2/HPF, less than about 1/HPF or less (e.g. 0) in the patient at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the therapeutic agent disclosed herein reduces peak eosinophil in at least one biopsy to less than about 14/HPF, less than about 13/HPF, less than about 12/HPF, less than about 11/HPF, less than about 10/HPF, less than about 9/HPF, less than about 8/HPF, less than about 7/HPF, less than about 6/HPF, less than about 5/HPF, less than about 4/HPF, less than about 3/HPF, less than about 2/HPF, less than about 1/HPF or less (e.g. 0) in the patient for about 1 week, about 1 month, about 2 months, about 3 months, about 4 months, about 5 months, about 6 months, about 1 year, about 2 years, about 5 years, or about 10 years, or more

In some embodiments, administration of the therapeutic agent according to the methods disclosed herein reduces peak eosinophil (per high power field (HPF) in at least one biopsy in a treated patient compared to peak eosinophil per HPF in an untreated patient or the same patient before treatment by at least about 10%, e.g., about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, inclusive of all values and subranges therebetween. In particular embodiments, the peak eosinophil count is reduced by an amount in the range of about 50% to about 99%, e.g., about 55%, about 60%, about 65%, about 70%, about 75%, about 80%, about 85%, about 90%, about 91%, about 92%, about 93%, about 94%, about 95%, about 96%, about 97%, about 98%, inclusive of all values and subranges therebetween.

In some embodiments, administration of the therapeutic agents disclosed herein reduces peak eosinophil in at least one biopsy from a treated patient at week 12, week 26, or week 52. In some embodiments, administration of the therapeutic agents disclosed herein reduces peak eosinophil in at least one biopsy from a treated patient to less than about 6/HPF at week 12, week 26, or week 52. In some embodiments, administration of the therapeutic agents disclosed herein reduces peak eosinophil in all tested biopsies from a treated patient at week 12, week 26, or week 52. In some embodiments, administration of the therapeutic agents disclosed herein reduces peak eosinophil in all tested biopsies from a treated patient to less than about 6/HPF at week 12, week 26, or week 52.

In some embodiments, administration of therapeutic agents disclosed herein reduce episodes of dysphagia and/or behavior modification (e.g. food avoidance, increase in liquid intake, etc.) in a treated patient. In some embodiments, reduction of episodes of dysphagia and/or behavior modification (e.g. food avoidance, increase in liquid intake, etc.) in a treated patient is measured by determining the number of episodes of dysphagia over a period of time, e.g., about two weeks. Such a measurement takes into consideration multiple occurrences of dysphagia occurring during the same day. In some embodiments, reduction of episodes of dysphagia and/or behavior modification (e.g. food avoidance, increase in liquid intake, etc.) in a treated patient is measured by determining Dysphagia-Free-Days in the patient. In some embodiments, improvement in Dysphagia-Free-Days in a patient is measured in conjunction with other patient measurements such as improved histologic scores (e.g. eosinophil counts) to measure patient response to treatment. In some embodiments, administration of the therapeutic agents disclosed herein reduce dysphagia and/or behavior modification (e.g. food avoidance, increase in liquid intake, etc.) in a treated patient compared with episodes of dysphagia and/or behavior modification (e.g. food avoidance, increase in liquid intake, etc.) in an untreated subject or in the same patient before treatment. In some embodiments, administration of the therapeutic agents disclosed herein reduce episodes of dysphagia and/or behavior modification (e.g. food avoidance, increase in liquid intake, etc.) to fewer than about 6 per week. In some embodiments, administration of the therapeutic agents disclosed herein reduces episodes of dysphagia and/or behavior modification (e.g. food avoidance, increase in liquid intake, etc.) to fewer than 6 per week over a time period of two weeks. In some embodiments, administration of the therapeutic agents disclosed herein reduces episodes of dysphagia and/or behavior modification (e.g. food avoidance, increase in liquid intake, etc.) to fewer than about 6 per week, about 5 per week, about 4 per week, about 3 per week, about 2 per week, about one per week, or none per week. In some embodiments, administration of the therapeutic agents disclosed herein reduces episodes of dysphagia and/or behavior modification (e.g. food avoidance, increase in liquid intake, etc.) to fewer than about 6 per week, about 5 per week, about 4 per week, about 3 per week, about 2 per week, about one per week, or none per week over a time period of two weeks.

In some embodiments, episodes of dysphagia and/or behavior modification (e.g. food avoidance, increase in liquid intake, etc.) are reduced by up to about 100%. In some embodiments, episodes of dysphagia and/or behavior modification (e.g. food avoidance, increase in liquid intake, etc.) are reduced by up to about 1%, about 5%, about 10%, about 20%, about 30%, about 40%, about 50%, about 60%, about 70%, about 80%, about 90%, or about 100% compared with episodes of dysphagia and/or behavior modification (e.g. food avoidance, increase in liquid intake, etc.) in an untreated patient or the same patient before treatment. In some embodiments, dysphagia and/or behavior modification (e.g. food avoidance, increase in liquid intake, etc.) is eliminated. In some embodiments, dysphagia and/or behavior modification (e.g. food avoidance, increase in liquid intake, etc.) is assessed in a treated patient between week 1 and year 10. In some embodiments, administration of the pharmaceutical compositions disclosed herein reduces episodes of dysphagia and/or behavior modification (e.g. food avoidance, increase in liquid intake, etc.) at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the pharmaceutical compositions disclosed herein reduces dysphagia and/or behavior modification (e.g. food avoidance, increase in liquid intake, etc.) for about 1 week, about 1 month, about 2 months, about 3 months, about 4 months, about 5 months, about 6 months, about 1 year, about 2 years, about 5 years, or about 10 years or more compared with the number of episodes of dysphagia and/or behavior modification (e.g. food avoidance, increase in liquid intake, etc.) in an untreated patient or the patient before treatment. In some embodiments, episodes of dysphagia and/or behavior modification (e.g. food avoidance, increase in liquid intake, etc.) are reduced by up to about 1%, about 5%, about 10%, about 20%, about 30%, about 40%, about 50%, about 60%, about 70%, about 80%, about 90%, or about 100% compared with episodes of dysphagia and/or behavior modification (e.g. food avoidance, increase in liquid intake, etc.) in an untreated patient or the same patient before treatment at about week 1, week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, episodes of dysphagia are reduced by up to about 1%, about 5%, about 10%, about 20%, about 30%, about 40%, about 50%, about 60%, about 70%, about 80%, about 90%, or about 100% compared with episodes of dysphagia and/or behavior modification (e.g. food avoidance, increase in liquid intake, etc.) in an untreated patient or the same patient before treatment for about 1 week, about 1 month, about 2 months, about 3 months, about 4 months, about 5 months, about 6 months, about 1 year, about 2 years, about 5 years, or about 10 years or more compared with the number of episodes of dysphagia and/or behavior modification (e.g. food avoidance, increase in liquid intake, etc.) in an untreated patient or the patient before treatment.

In some embodiments, administration of the pharmaceutical compositions disclosed herein reduces episodes of dysphagia and/or behavior modification (e.g. food avoidance, increase in liquid intake, etc.) in a treated patient at week 12, week 26, or week 52 compared with episodes of dysphagia and/or behavior modification (e.g. food avoidance, increase in liquid intake, etc.) in an untreated patient or the same patient before treatment.

In some embodiments, administration of the therapeutic agents disclosed herein reduces food impaction in a treated patient compared with episodes of food impaction in an untreated patient or in the same patient before treatment as measured using the PRO assessment questionnaire described herein (e.g., the daily diary and/or the 24 hour diary). In some embodiments, administration of the therapeutic agents disclosed herein reduces episodes of food impaction to fewer than 4 per week. In some embodiments, administration of the therapeutic agents disclosed herein reduces episodes of food impaction to fewer than 4 per week over a time period of two weeks. In some embodiments, administration of the therapeutic agents disclosed herein reduces episodes of food impaction to fewer than about 4 per week, about 3 per week, about 2 per week, about one per week, or none per week. In some embodiments, administration of the therapeutic agents disclosed herein reduces episodes of food impaction to fewer than about 4 per week, about 3 per week, about 2 per week, about one per week, or none per week over a time period of two weeks.

In some embodiments, episodes of food impaction are reduced by up to about 100%. In some embodiments, episodes of food impaction are reduced by up to about 10%, about 5%, about 10%, about 20%, about 30%, about 40%, about 50%, about 60%, about 70%, about 80%, about 90%, or about 100% compared with episodes of food impaction in an untreated patient or the same patient before treatment. In some embodiments, food impaction is eliminated. In some embodiments, episodes of food impaction are assessed in a treated patient between week 1 and year 10. In some embodiments, administration of the therapeutic agents disclosed herein reduces episodes of food impaction at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the therapeutic agents disclosed herein reduces episodes of food impaction for about 1 week, about 1 month, about 2 months, about 3 months, about 4 months, about 5 months, about 6 months, about 1 year, about 2 years, about 5 years, or about 10 years or more compared with the number of episodes of food impaction in an untreated patient or the same patient before treatment. In some embodiments, episodes of food impaction are reduced by up to about 10%, about 5%, about 10%, about 20%, about 30%, about 40%, about 50%, about 60%, about 70%, about 80%, about 90%, or about 100% compared with episodes of food impaction in an untreated patient or the same patient before treatment at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, episodes of food impaction are reduced by up to about 1%, about 5%, about 10%, about 20%, about 30%, about 40%, about 50%, about 60%, about 70%, about 80%, about 90%, or about 100% compared with episodes of food impaction in an untreated patient or the same patient before treatment for about 1 week, about 1 month, about 2 months, about 3 months, about 4 months, about 5 months, about 6 months, about 1 year, about 2 years, about 5 years, or about 10 years or more compared with the number of episodes of food impaction in an untreated patient or the same patient before treatment.

In some embodiments, administration of the therapeutic agents disclosed herein reduces episodes of food impaction in a treated patient at week 12, week 26, or week 52 compared with food impaction in an untreated patient or the same patient before treatment.

In some embodiments, administration of the therapeutic agents disclosed herein improves characteristics as measured by endoscopy (e.g. EndoFlip) in a treated patient compared with an untreated patient or the same patient before treatment commenced. These characteristics include, but are not limited to esophagus diameter, esophageal compliance, focal narrowing of the esophagus, esophageal body distensibility, esophageal body cross-sectional areas (CSA), and intra-luminal diameter. In some embodiments, the histology characteristics do not improve, but the patient records an improvement in the episodes of dysphagia on the PRO in Appendix A.

In some embodiments, the characteristics as measured by endoscopy are assessed in a treated patient between week 1 and year 10. In some embodiments, administration of the therapeutic agents disclosed herein improves characteristics as measured by endoscopy at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3 compared with an untreated patient or the same patient before treatment. In some embodiments, administration of the therapeutic agents disclosed herein improves characteristics as measured by endoscopy for about 1 week, about 1 month, about 2 months, about 3 months, about 4 months, about 5 months, about 6 months, about 1 year, about 2 years, about 5 years, or about 10 years or more compared with an untreated patient or the same patient before treatment. In some embodiments, characteristics as measured by endoscopy are improved by up to about 100%. In some embodiments, characteristics as measured by endoscopy are improved by up to about 1%, about 5%, about 10%, about 20%, about 30%, about 40%, about 50%, about 60%, about 70%, about 80%, about 90%, or about 100% compared with an untreated patient or the same patient before treatment. In some embodiments, administration of the therapeutic agents disclosed herein improves characteristics as measured by endoscopy by up by about 1%, about 5%, about 10%, about 20%, about 30%, about 40%, about 50%, about 60%, about 70%, about 80%, about 90%, or about 100% compared with an untreated patient or the same patient before treatment at about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the therapeutic agents disclosed herein improves characteristics as measured by endoscopy by up by about 1%, about 5%, about 10%, about 20%, about 30%, about 40%, about 50%, about 60%, about 70%, about 80%, about 90%, or about 100% compared with EndoFlip score in an untreated patient or the same patient before treatment for about 1 week, about 1 month, about 2 months, about 3 months, about 4 months, about 5 months, about 6 months, about 1 year, about 2 years, about 5 years, or about 10 years or more.

In some embodiments, administration of the therapeutic agents disclosed herein improves characteristics as measured by endoscopy in a treated patient at week 12, week 26, or week 52 compared with an untreated patient or the same patient before treatment.

In some embodiments, administration of the pharmaceutical compositions disclosed reduces the number of episodes associated with EoE experienced by a patient over a period of time. Non-limiting examples of such episodes include difficulty swallowing a pill or food. The occurrence of such episodes may be reported by the patient as a feeling of discomfort after swallowing a pill or food, and may be measured after each instance of swallowing a pill or food or over a 24 hour period or more. In some embodiments, the number of episodes is measured using the daily diary assessment described in FIG. 1 and/or the 24 hour diary described in FIG. 2).

In some embodiments, the number of episodes occurring over said period of time is reduced by at least 1 episode, at least 2 episodes, at least 3 episodes, at least 4 episodes, at least 5 episodes, at least 6 episodes, at least 7 episodes, at least 8 episodes, at least 9 episodes, at least 10 episodes, at least 11 episodes, at least 12 episodes, at least 13 episodes, at least 14 episodes, at least 15 episodes, at least 16 episodes, at least 17 episodes, at least 18 episodes, at least 19 episodes, or at least 20 episodes, least 21 episodes, at least 22 episodes, at least 23 episodes, at least 24 episodes, at least 25 episodes, at least 26 episodes, at least 27 episodes, at least 28 episodes, at least 29 episodes, or at least 30 episodes, least 31 episodes, at least 32 episodes, at least 33 episodes, at least 34 episodes, at least 35 episodes, at least 36 episodes, at least 37 episodes, at least 38 episodes, at least 39 episodes, or at least 40 episodes, least 41 episodes, at least 42 episodes, at least 43 episodes, at least 44 episodes, at least 45 episodes, at least 46 episodes, at least 47 episodes, at least 48 episodes, at least 49 episodes, or at least 50 episodes. In some embodiments, the time period is about 1 week, about 2 weeks, about 3 weeks, about 4 weeks, about 5 weeks, about 6 weeks, about 7 weeks, about 8 weeks, about 9, weeks about 10 weeks, about 20 weeks, about 30 weeks, about 40 weeks, about 50 weeks, about 60 weeks, about 70 weeks, about 80 weeks, about 90 weeks, about 100 weeks, about 1 year, about 2 years, or about 3 years.

### Purpose-Bound Products

The PRO assessment and methods of treating and monitoring of the present disclosure provide methods of selecting or identifying patient populations for treatment. In some embodiments, the disclosure provides for a therapeutic agent (as described herein e.g. a corticosteroid or antibody) for use in a method of treating dysphagia characterized in that the patient has a mean score from about 2 and about 7 as measured in the PRO assessment. In some embodiments, the therapeutic agent is used in a method of treating dysphagia, characterized in that the patient has been selected to have a mean score from about 2 to about 7 as measured in the PRO assessment. In some embodiments, the therapeutic agent is used in a method of treating dysphagia, wherein the method comprises determining whether methods of the present disclosure includes determining whether a patient has a score between about 2 and 7 as measured in the PRO assessment and treating the patient with the therapeutic agent if so. The means to determine if the patient has a mean score falling with the treatment score (i.e., between 2 and 7) using the PRO assessment is described herein. For example, in some embodiments, the PRO assessment includes (i) at least one question determining the severity of the dysphagia event, as the event occurs; (ii) at least one question determining the pain associated with the dysphagia event, as the event occurs; and (iii) at least one question determining the discomfort associated with the dysphagia event, as the event occurs; wherein the severity question is scored from 0 to 10; the pain question is scored from 0 to 10; and the discomfort question is scored from 0 to 10; and the answers to said questions to determine a score calculated over 1-21 days, and the daily average score is calculated daily. In some embodiments, the score is calculated over 14 days.

### Pharmaceutical Compositions

Any therapeutic agent or therapy, including but not limited to diet changes, proton pump inhibitors (PPI), dilating strictures, and/or therapeutic agents, which can be used to treat or ameliorate dysphagia can be used in the methods described herein. In some embodiments, any therapeutic agent which can be used to treat or ameliorate inflammation of the upper gastrointestinal tract (e.g., eosinophilic esophagitis) can be used in the methods described herein. Suitable therapeutic agents include those that reduce esophageal inflammation, reduce the number of esophageal eosinophils, or a combination thereof.

In some embodiments, the therapeutic agents disclosed herein are co-administered with one or more corticosteroids. Suitable corticosteroids include, but are not limited to hydrocortisone, prednisone, prednisolone, methylprednisolone, dexamethasone, betamethasone, etc. or mineralocorticoid potencies (e.g., alsosterone), budesonide, fluticasone, flunisolide, ciclesonide, mometasone, beclomethasone, tixocortol and salts, or esters and mixtures thereof.

In some embodiments, therapeutic agents disclosed herein are co-administered with one or more proton pump inhibitors (PPI). Suitable PPIs include, but are not limited to, omeprazole, lansoprazole, dexlansoprazole, rabeprazole, pantoprazole, and esomeprazole. In some embodiments the PPI is administered at high doses.

The one or more therapeutic agents may be “co-administered”, i.e., administered together in a coordinated fashion to a subject, either as separate pharmaceutical compositions or admixed in a single pharmaceutical composition. By “co-administered”, the one or more therapeutic agents may also be administered simultaneously with the present pharmaceutical compositions, or be administered separately, including at different times and with different frequencies. The one or more therapeutic agents may be administered by any known route, such as orally, intravenously, intramuscularly, nasally, subcutaneously, intra-vaginally, intra-rectally, and the like; and the therapeutic agent may also be administered by any conventional route.

In some embodiments, the therapeutic agent comprises one or more immunosuppressants. Suitable immunosuppressants include, but are not limited to, cyclosporine, tacrolimus, prednisolone, hydrocortisone, sirolimus, everolimus, azathioprine, mycophenolic acid, methotrexate, basiliximab, daclizumab, rituximab, mepolizumab (anti-IL-5), reslizumab (anti-IL-5), QAX576 (anti-IL-13), omalizumab (anti-immunoglobulin-E), infliximab (anti-TNF-α), anti-thymocyte globulin, and anti-lymphocyte globulin.

In some embodiments, the pharmaceutical compositions disclosed herein are co-administered with one or more antibodies. Suitable anti-bodies include, include IL-4, IL-5, and IL-13 antibodies. Non-limiting examples include basiliximab, daclizumab, rituximab, mepolizumab (anti-IL-5), reslizumab (anti-IL-5), QAX576 (anti-IL-13), and omalizumab (anti-immunoglobulin-E).

Therapeutic agents in the above paragraphs can be combined. When two or more medicines are used in combination, dosage of each medicine is commonly identical to the dosage of the medicine when used independently, but when a medicine interferes with metabolism of other medicines, the dosage of each medicine is properly adjusted. Each medicine may be administered simultaneously or separately in an appropriate time interval.

The therapeutic agent for use in the present methods can be formulated into any appropriate dosage form, such as oral orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques.

In some embodiments, the pharmaceutical compositions used in (or for use in) the methods described herein can be any dosage form which can be used to topically administer a therapeutic agent (e.g., corticosteroid) to the esophagus. Non-limiting examples of suitable dosage forms include liquid compositions (e.g., solutions, suspensions, and slurries), gels, and solid compositions which form a liquid or gel after oral administration. For example, orally disintegrating compositions (e.g., ODT, effervescent, film, lyophilize matrix, or wafer), lozenges, and lollipops can from a solution, suspension, or gel comprising the therapeutic agent in the oral cavity of the patient, and after the solution or suspension is swallowed, the corticosteroid dissolved or suspended therein contacts the esophagus as the liquid traverses the esophageal tract. In a preferred embodiment, the pharmaceutical composition is in the form of an ODT.

In some embodiments, the corticosteroid used in the compositions and methods described herein is a topically acting corticosteroid. In some embodiments, the corticosteroid has low or substantially no systemic effect. In some embodiments, corticosteroids that have low or no systemic effects are those which have no significant systemic glucocorticoid or mineralocorticoid activity after oral administration in humans. Corticosteroid with “no significant systemic glucocorticoid or mineralocorticoid activity after oral administration in humans” refer to corticosteroids, or pharmaceutical compositions comprising corticosteroids, which have less than about 20% systemic glucocorticoid or mineralocorticoid activity after oral administration, e.g., less than about 15%, less than about 10%, less than about 5%, less than about 5%, less than about 4%, less than about 3%, less than about 2%, or less than about 1%. Systemic glucocorticoid or mineralocorticoid activity can be determined using methods known in the art, such as by measuring morning cortisol levels.

In some embodiments, corticosteroids for use in the methods and compositions described herein have a systemic bioavailability of less than or equal to about 20% of the administered dose. Non-limiting examples of oral corticosteroids that have a bioavailability of less than or equal to about 20% include fluticasone, flunisolide, budesonide, circlesone, mometasone, tixocortol, and beclomethasone, and pharmaceutically acceptable salts, solvates, esters, polymorphs or prodrugs thereof. In preferred embodiments, the oral corticosteroid used in the methods and compositions described herein is fluticasone propionate.

Wafers can include dried or lyophilized compositions such as orally disintegrating or dissolving dosage forms prepared using Zydis® lyophilization technology (e.g., as described in U.S. Pat. No. 6,316,027), containing a corticosteroid as the active pharmaceutical ingredient. Film dosage forms can include edible films such as those described in U.S. Pat. No. 6,596,298 or U.S. Pat. No. 6,740,332, containing a corticosteroid as the active pharmaceutical ingredient. In some embodiments, the solid composition comprises a lyophilized matrix, wherein the lyophilized matrix comprises corticosteroid, the carrier and excipient. Suitable excipients include, but are not limited to, mannitol, xylitol, sorbitol, maltol, maltitol, lactose, sucrose, maltose, and combinations thereof.

Effervescent tablets and effervescent orally dispersing tablets can include those disclosed in U.S. Pat. Nos. 9,867,780 and 8,580,300. Such formulations contain weak acids or salts of weak acids, such as tartaric acid, acetic acid, lactic acid, or citric acid, or pharmaceutically acceptable salts thereof, such as magnesium, calcium, or sodium salts. These formulations may also include pharmaceutically acceptable excipients that release CO₂ upon contact with water (e.g., saliva), such as carbonic acid, and salts of carbonates and bicarbonates, such as sodium and potassium salts. In some embodiments, such effervescent tablets are formulated to dissolve in a solution prior to oral administration. Such formulations may further comprise polyvinylpyrrolidone.

### Dosing and Administration

The therapeutic agents disclosed herein may be administered in any appropriate dose and using any therapeutic agent. While one of skill in the art can determine the desirable dose in each case, a suitable dose of the therapeutic agent for achievement of therapeutic benefit, may, for example, be in a range of about 1 microgram (μg) to about 100 milligrams (mg) per kilogram body weight of the recipient per day, preferably in a range of about 10 μg to about 50 mg per kilogram body weight per day and most preferably in a range of about 10 μg to about 50 mg per kilogram body weight per day. In some embodiments, the therapeutic agents is administered at a low dose, e.g., about 20 mg or less. In some embodiments, the oral corticosteroid is administered at about 1 mg per kilogram body weight per day, about 3 mg per kilogram body weight per day, and/or about 9 mg per kilogram body weight per day. The desired dose may be presented as one dose or two or more sub-doses administered at appropriate intervals throughout the day. These sub-doses can be administered in unit dosage forms, for example, containing from about 10 μg to about 1000 mg. In some embodiments, the pharmaceutical composition is administered in a unit dosage form of 0.75 mg, 1.5 mg, 3.0 mg, 4.5 mg, 6.0 mg, or 7.5 mg.

In some embodiments, the therapeutic agent described herein (e.g., a liquid or solid composition comprising an oral corticosteroid) is administered to a patient once a day at bedtime (HS). In some embodiments, the therapeutic agent is administered to a patient twice a day (BID). In some embodiments, the therapeutic agent is administered to a patient once in the morning and once in the evening. In some embodiments, the therapeutic agent is administered on an empty stomach (e.g. at least 2 hours after eating or at least 1 hour before eating; or at least 30 minutes before or after eating). In some embodiments, the therapeutic agent is administered to a patient 30 minutes before breakfast and 30 minutes before bedtime. In some embodiments, administering the pharmaceutical composition before bedtime decreases systemic adsorption of the oral corticosteroid compared to systemic adsorption observed after daytime dosing.

Thus, in some embodiments, the pharmaceutical composition is administered during the evening between about 7 pm and about 10 pm, e.g., at about 7 pm, 7:30 pm, about 8 pm, about 8:30 pm, about 9 pm, or about 9:30 pm, inclusive of all values and subranges therebetween. In some embodiments, the pharmaceutical composition is administered about 30 minutes before the target sleep time. The term “target sleep time” can mean the time of day that the patient anticipates going to bed.

Accordingly, in particular embodiments, the therapeutic agent is administered once daily, at nighttime while the patient is lying down (or wherein the patient lays down immediately after oral administration). In other particular embodiments, the therapeutic agent is administered twice daily, wherein the patient may remain upright during the first daily dose and the patient is lying down for the second daily dose (or the patient lays down immediately after oral administration). In further embodiments, the patient is lying down for both daily doses.

In various embodiments, the therapeutic agent is administered HS while the patient is lying down (or the patient lays down immediately following administration). In other embodiments, the therapeutic agent is administrated during daytime (e.g., BID or QD administration), while the patient is lying down (or the patient lays down immediately following administration). In various embodiments, the patient remains lying down following administration for an amount of time sufficient for topical deposition and/or contact of the therapeutic agent onto the esophagus to treat inflammation thereof (e.g., a time sufficient to result in improvement of EoE using the measurements described herein, such as a reduction in episodes of dysphagia). In some such embodiments, the patient remains lying down following administration for an amount of time in the range of from about 1 minute to about 8 hours, including about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hr, about 1.1 hr, about 1.2 hr, about 1.3 hr, about 1.4 hr, about 1.5 hr, about 1.6 hr, about 1.7 hr, about 1.8 hr, or about 1.9 hr, about 2 hrs, about 2.1 hr, about 2.2 hr, about 2.3 hr, about 2.4 hr, about 2.5 hr, about 2.6 hr, about 2.7 hr, about 2.8 hr, or about 2.9 hr, about 3 hrs, about 3.1 hr, about 3.2 hr, about 3.3 hr, about 3.4 hr, about 3.5 hr, about 3.6 hr, about 3.7 hr, about 3.8 hr, or about 3.9 hr, about 4 hrs, about 4.1 hr, about 4.2 hr, about 4.3 hr, about 4.4 hr, about 4.5 hr, about 4.6 hr, about 4.7 hr, about 4.8 hr, about 4.9 hr, about 5 hrs, about 5.1 hr, about 5.2 hr, about 5.3 hr, about 5.4 hr, about 5.5 hr, about 5.6 hr, about 5.7 hr, about 5.8 hr, about 5.9 hr about 6 hrs, about 6.1 hr, about 6.2 hr, about 6.3 hr, about 6.4 hr, about 6.5 hr, about 6.6 hr, about 6.7 hr, about 6.8 hr, or about 6.9 hr, about 7 hrs, about 7.1 hr, about 7.2 hr, about 7.3 hr, about 7.4 hr, about 7.5 hr, about 7.6 hr, about 7.7 hr, about 7.8 hr, or about 7.9 hr, inclusive of all values and subranges therebetween. In embodiments in which the therapeutic agent is administered during daytime, the patient remains lying down for an amount of time in the range of from about 1 minute to about 60 minutes, including about 5 minutes, about 10 minutes, about 15 minutes, about 20 minutes, about 25 minutes, about 30 minutes, about 35 minutes, about 40 minutes, about 45 minutes, about 50 minutes, about 55 minutes, inclusive of all values and subranges therebetween. In certain embodiments, the patient remains lying down for about 5 to about 10 minutes.

As used herein, “lying down,” “lays down,” and derivations and variants thereof, refer to a patient adopting a supine, prone, or laterally recumbent position, as on a bed or on the ground, or a substantially horizontal body position, whereby the corticosteroid (upon swallowing) contacts the esophagus and topically deposits the corticosteroid on esophagus, e.g., at the site of inflammation. As used herein, “substantially horizontal” refers to a body position which at least 10° less than vertical, e.g., less than about 15°, less than about 20°, less than about 25°, less than about 30°, less than about 35°, less than about 40°, less than about 45°, less than about 50°, less than about 55°, less than about 65°, less than about 70°, less than about 75°, less than about 80°, less than about 85°, or about 90° from vertical, inclusive of all values and ranges therebetween. For example, when the therapeutic agent is formulated as an ODT, the ODT rapidly disintegrates in the mouth of the supine patient to form a suspension comprising the therapeutic agent which is swallowed. The suspension then traverses the esophagus of the patient, providing topical contact of the therapeutic agent on the esophagus to topically treat inflammation thereof. As used herein, “upright” refers to a patient adopting essentially any other position, including, but not limited to, standing or sitting.

In some embodiments, the therapeutic agent is administered to a patient at bedtime. In some embodiments, the therapeutic agent is administered to a patient at bedtime while the patient is lying down. In some embodiments, the therapeutic agent is administered to the patient while lying down and prior to sleep (e.g., about 1 minute to about 1 hour before going to sleep, e.g., about 1 minute, about 5 minutes, about 10 minutes, about 15 minutes, about 20 minutes, about 25 minutes, about 30 minutes, about 35 minutes, about 40 minute, about 45 minutes, about 50 minutes, about 55 minutes, inclusive of all values therein). In preferred embodiments, the therapeutic agent is administered to a lying down patient about 30 minutes before bedtime. In some embodiments, the therapeutic agent is administered to a patient after the evening meal, e.g., from about 1 minute to about 5 hours after the evening meal (e.g., about 5 minutes, about 10 minutes, about 15 minutes, about 20 minutes, about 25 minutes, about 30 minutes, about 35 minutes, about 40 minute, about 45 minutes, about 50 minutes, about 55 minutes, about 1 hour, about 1.5 hours, about 2 hours, about 2.5 hours, about 3 hours, about 3.5 hours, about 4 hours, about 4.5 hours, inclusive of all values and subranges therebetween. In preferred embodiments, the therapeutic agent is administered at least about 30 minutes after the evening meal.

In some embodiments, the therapeutic agent is administered to a patient at least about 2 hours after the evening meal (with no snacks) while the patient is lying down. In some embodiments, the therapeutic agent is administered to a patient at least about 4 hours after the evening meal (with no snacks) while the patient is lying down. In some embodiments, the therapeutic agent is administered to a patient within about 2 hours after the evening meal (with no snacks) while the patient is lying down. In some embodiments, the therapeutic agent is administered to a patient within about 4 hours after the evening meal (with no snacks) while the patient is lying down. In some embodiments, after administration of the therapeutic agent while the patient is lying down, the patient goes to sleep. In some embodiments, after administration of the therapeutic agent while the patient is lying down, the patient does not rise for at least one hour.

In some embodiments, the pharmaceutical composition is administered to a patient from one to five times a day. In some embodiments, the pharmaceutical composition is administered to a patient at least once a day, at least twice a day, at least three times a day, at least 4 times a day, or at least five times a day. In some embodiments, the pharmaceutical composition is administered to a patient at least one to five times a day for one week to 10 years or more. In some embodiments, the pharmaceutical composition is administered to a patient at least once a day, at least twice a day, at least three times a day, at least 4 times a day, or at least five times a day for at least one week, at least two weeks, at least three weeks, at least four weeks, at least five weeks, at least six weeks, at least seven weeks, at least eight weeks, at least nine weeks, at least ten weeks, at least fifteen weeks, at least twenty weeks, at least thirty weeks, at least forty weeks, at least fifty weeks, at least fifty-two weeks, at least sixty weeks, at least seventy weeks, at least eighty weeks, at least ninety weeks, or at least one hundred weeks or more. In some embodiments, the pharmaceutical composition is administered to a patient indefinitely. In some embodiments, the pharmaceutical composition is administered twice a day for at least about 6 weeks, at least about 8 weeks, at least about 10 weeks, or at least about 12 weeks. In some embodiments, the pharmaceutical composition is administered twice a day during the induction and/or maintenance phase. In some embodiments, the pharmaceutical composition is administered twice a day for at least about 6 weeks, at least about 8 weeks, at least about 10 weeks, or at least about 12 weeks during the induction phase.

In some embodiments, the therapeutic agent is administered to a patient at the same dose multiple times a day. In some embodiments, the therapeutic agent is administered to a patient at the same dose at least twice a day, at least three times a day, at least 4 times a day, or at least five times a day. In some embodiments, the therapeutic agent is administered to the patient at the some dose throughout the course of treatment, irrespective of an improvement the score as measured using the PRO questionnaire. In some embodiments, the therapeutic agent is administered to the patient at a reduced dose after a reduction in the score is recorded on the PRO questionnaire. In some embodiments, the therapeutic agent is administered to a patient at the same dose two to five times a day for one week to 10 years or more (i.e. for continuous treatment). In some embodiments, the therapeutic agent is administered to a patient at least at the same dose at least twice a day, at least three times a day, at least 4 times a day, or at least five times a day for at least one week, at least two weeks, at least three weeks, at least four weeks, at least five weeks, at least six weeks, at least seven weeks, at least eight weeks, at least nine weeks, at least ten weeks, at least fifteen weeks, at least twenty weeks, at least thirty weeks, at least forty weeks, at least fifty weeks, at least fifty-two weeks, at least sixty weeks, at least seventy weeks, at least eighty weeks, at least ninety weeks, or at least one hundred weeks or more or indefinitely.

In some embodiments, the therapeutic agent is administered twice a day at different doses. In some embodiments, the therapeutic agent is administered twice a day, with the morning dose being greater than the evening dose. In some embodiments, the therapeutic agent is administered twice a day, with the morning dose being less than the evening dose.

In some embodiments, the patient is administered different doses of the therapeutic agent depending on the phase of the regimen. For example, the regimen may be divided into at least induction, treatment, withdrawal (i.e., drug holiday), or maintenance phase. In some embodiments, the regimen includes at least one of these phases. In some embodiments, the regimen includes a combination of one or more of these phases. In some embodiments, the regimen includes all of these phases.

In some embodiments, the regimen includes induction and withdrawal. In some embodiments, the regimen includes multiple cycles of induction and withdrawal as needed. In some embodiments, the regimen includes multiple cycles of induction and withdrawal repeated indefinitely. In some embodiments, the induction period does not result in a recurrence of symptoms.

The regimen phases may be any appropriate duration. In some embodiments, the induction phase lasts between about 1 and about 10 weeks, about 12 weeks, about 15 weeks, about 20 weeks, about 30 weeks, about 40 weeks, or about 50 weeks. In some embodiments, the induction phase lasts about 14 weeks. In some embodiments, the withdrawal phase lasts between about 1 and about 10 weeks, about 15 weeks, about 20 weeks, about 30 weeks, about 40 weeks, about 50 weeks, about 1 year, about 2 years, about 5 years, about 10 years or indefinitely. In some embodiments, the withdrawal phase lasts until symptoms recur. In some embodiments, the withdrawal phase lasts about 14 weeks. In some embodiments, the maintenance phase lasts between about 1 and about 15 weeks, about 20 weeks, about 30 weeks, about 40 weeks, about 50 weeks, about 1 year, about 2 years, about 5 years, about 10 years or more. In some embodiments, the maintenance phase lasts about 28 weeks. In some embodiments, the maintenance phase is an indefinite duration.

In some embodiments, the patient is administered a greater dose in one or more regimen phases compared to the others. In some embodiments, the patient is administered the same dose in one or more regimen phases. In some embodiments, the patient is administered the same dose in every regimen phases. In some embodiments, the patient is administered no dose during one or more phases.

In some embodiments, the patient is administered a greater dose during the induction stage compared to the maintenance stage. In some embodiments, the patient is administered a smaller dose during the induction stage compared to the maintenance stage. In some embodiments, the patient is administered no dose during either the induction or maintenance stage. In some embodiments, the patient is administered no dose during both the induction and maintenance stages. In some embodiments, the patient is administered the same dose during the induction and maintenance stages. In some embodiments, the patient is administered substantially the same dose during the induction and maintenance stages. For example, when the therapeutic agent is a corticosteroid, the patient is administered 3.0 mg BID during the induction stage, and 1.5 mg BID during the maintenance stage. In some embodiments, the patient is administered 3.0 mg BID during the induction stage, and 1.5 mg HS during the maintenance stage. In some embodiments, the patient is administered 1.5 mg BID during the induction stage, and 3.0 mg BID during the maintenance stage. In some embodiments, the patient is administered 1.5 mg HS during the induction stage, and 3.0 mg BID during the maintenance stage. In some embodiments, the patient is administered 1.5 mg BID during both the induction and maintenance stages. In some embodiments, the patient is administered 1.5 mg HS during the induction and maintenance stages. In some embodiments, the patient is administered 3.0 mg BID during the induction and maintenance stages. In some embodiments, the patient is administered 6.0 mg BID during the induction stage, and 3.0 or 1.5 mg BID during the maintenance stage. In some embodiments, the patient is administered 6.0 mg BID during the induction stage, and 3.0 or 1.5 mg HS during the maintenance stage. In some embodiments, the patient is administered 1.5 or 3.0 mg BID during the induction stage, and 6.0 mg BID during the maintenance stage. In some embodiments, the patient is administered 1.5 or 3.0 mg HS during the induction stage, and 6.0 mg BID during the maintenance stage. In some embodiments, the patient is administered 6.0 or 3.0 mg BID during both the induction and maintenance stages. In some embodiments, the patient is administered 6.0 or 3.0 mg HS during the induction and maintenance stages. In some embodiments, the patient is administered 6.0 mg BID during the induction and maintenance stages.

In certain embodiments, the patient is a human, but in other embodiments may be a non-human mammal, such as a domesticated pet (e.g., dog or cat), or livestock or farm animal (e.g., horse, cow, sheep, or pig).

### Patient Populations

Any patient diagnosed with, or presumed to be suffering from an inflammatory gastrointestinal disorder, may be administered the pharmaceutical compositions of the present disclosure. In some embodiments, the patient is an adult. In some embodiments, the patient is an adolescent. In some embodiments, the patient is a child. In some embodiments, the patient is an infant.

In some embodiments, the inflammatory gastrointestinal disorder is EoE. The patient may be diagnosed using any appropriate measures in the art. In some embodiments, the patient is diagnosed with EoE based on symptoms, score in the assessment in Appendix A (e.g., at least 3 episodes of dysphagia per week for at least 2 weeks), histology, and/or failed documentation on proton pump inhibitors. In some embodiments, the patient received PPI therapy prior to administration of a therapeutic agent of the present disclosure. In some embodiments, the patient did not receive PPI therapy prior to administration of a therapeutic agent of the present disclosure. In some embodiments, the patient failed to improve after 8 weeks of high-dose (e.g. 40 mg) PPI. A lack of response to PPI therapy may be defined as Peak eosinophil count ≥15/HPF in at least one biopsied location after 8 weeks of treatment with a high dose PPI. In some embodiments, the failure of PPI therapy is documented before administration of a pharmaceutical composition of the present disclosure. In some embodiments, the failure of PPI therapy is documented subsequently to administration of a pharmaceutical composition of the present disclosure. In some embodiments, patients which did not respond to previous PPI therapy are administered a high dose of the oral corticosteroid according to (or for use in) the methods disclosed herein, such as 6.0 mg, 7.5 mg, or more (e.g., about 9.0 mg to about 20 mg, including about 9 mg, about 10 mg, about 11 mg, about 12 mg, about 13 mg, about 14 mg, about 15 mg, about 16 mg, about 17 mg, about 18 mg, and about 19 mg, inclusive of all values and subranges therebetween).

In some embodiments, if the patient is a child or adolescent receiving corticosteroid therapy, treatment may include randomized withdrawal to characterize the persistence of the treatment effect, the incidence of relapse, and/or the need for redosing. In some embodiments, pediatric patients (e.g. child or adolescent) will be monitored for signs of glucocorticoid excess.

In some embodiments, the patient diagnosed with EoE has an esophageal stricture. In some embodiments, said patient is administered a high dose of the oral corticosteroid according to (or for use in) the methods disclosed herein, such as 6.0 mg, 7.5 mg, or more (e.g., about 9.0 mg to about 20 mg, including about 9 mg, about 10 mg, about 11 mg, about 12 mg, about 13 mg, about 14 mg, about 15 mg, about 16 mg, about 17 mg, about 18 mg, and about 19 mg, inclusive of all values and subranges there between).

In some embodiments, the patient diagnosed with EoE has a severe food allergy (e.g., a lactose or starch allergy). In some embodiments, said patient is administered a high dose of the oral corticosteroid according to (or for use in) the methods disclosed herein, such as 6.0 mg, 7.5 mg, or more (e.g., about 9.0 mg to about 20 mg, including about 9 mg, about 10 mg, about 11 mg, about 12 mg, about 13 mg, about 14 mg, about 15 mg, about 16 mg, about 17 mg, about 18 mg, and about 19 mg, inclusive of all values and subranges therebetween).

In some embodiments, the patient has been diagnosed with EoE by histological analysis. In some embodiments, the patient is diagnosed as having ≥15 peak eosinophil count per HPF (400× magnification) in at least one biopsy. In some embodiments, there are at minimum of 6 biopsies taken from the patient. In some embodiments, at least 3 biopsies are taken from each of the proximal and the distal esophagus.

In some embodiments, the patient is diagnosed as having EoE by their score in the assessment listed in Appendix A and/or measurement of esophageal characteristics via endoscopy (e.g. EndoFlip).

In some embodiments, the patient is diagnosed as having EoE based on symptoms, including but not limited to episodes of food impaction, episodes of food impaction requiring endoscopy, food avoidance, vomiting, reflux, and/or dysphagia. In some embodiments, the patient is diagnosed as having EoE based on dysphagia (difficulty swallowing). In some embodiments, the patient is diagnosed as having EoE based on experiencing dysphagia at least 3 times per week within 2 weeks. In some embodiments, the patient is diagnosed as having EoE based on experiencing at least 3 episodes of dysphagia occurring per week for each of two Baseline Symptom Assessments using the assessment listed in Appendix A.

Patient outcome and response to administration with a pharmaceutical composition of the present disclosure may be monitored or measured using any appropriate means in the art (e.g. endoscopy, histology, questionnaires).

Patients who exhibit an improvement of symptoms and/or histologic response after treatment commences are categorized as Responders. In some embodiments, patients who exhibit <15 peak eosinophils/HPF are categorized as Responders. In some embodiments, patients who exhibit <6 peak eosinophils/HPF are categorized as Responders. In some embodiments, patients who exhibit <6 peak eosinophils/HPF and no worsening of symptoms (e.g. no increase in assessment score compared to baseline; stricture requiring dilation) are categorized as Responders. In some embodiments, patients who exhibit <6 peak eosinophils/HPF and an improvement of symptoms (e.g. improvement in assessment score compared to baseline; stricture requiring dilation) are categorized as Responders. In some embodiments, improvement in assessment score is fewer than 6 episodes of dysphagia over a 14-day period. In some embodiments, patients who exhibit <6 peak eosinophils/HPF and no episodes of food impaction are categorized as Responders. In some embodiments, Responders exhibit evidence of inflammatory endoscopic remission such as an absence of white exudate and/or furrows. In some embodiments, Responders exhibit evidence of fibrotic remission including an absence of strictures and rings or moderate to severe rings. In some embodiments, Responders exhibit improved vascularity. In some embodiments, Responders exhibit improved biomarkers (e.g. IL-5, IgE levels).

In some embodiments, patients who are categorized as Responders enter the maintenance stage of the regimen. In some embodiments, patients who are categorized as Responders are administered a different dose of a pharmaceutical composition of the present disclosure after categorization. In some embodiments, Responders receive a greater dose after categorization after categorization. In some embodiments, Responders receive a smaller dose after categorization. In some embodiments, Responders receive the same dose after categorization. In some embodiments, Responders receive substantially the same dose after categorization.

In some embodiments, the patient is classified as a responder if the therapeutic agents disclosed herein are administered in an induction phase and a maintenance phase, and during the induction phase improvement in Peak eosinophilic counts in at least one esophageal biopsy and/or at least no worsening of PRO scores are observed, and where the maintenance phase comprises a dose at least equal to, more than or less than the induction phase.

Patients who do not meet the definition of Responder as disclosed above are categorized as Non-Responders. Patients whose histologic score and/or symptoms worsen are categorized as Relapse. In some embodiments, patients whose histologic score and/or symptoms worsen at any point during treatment are categorized as Relapse. In some embodiments, patients who experience food impaction requiring endoscopy and/or clinically-significant worsening of symptoms are categorized as Relapse. In some embodiments, patients who are categorized as Non-Responders or Relapsers are administered a different dose of a therapeutic of the present disclosure after categorization. In some embodiments, Non-Responders and/or Relapsers receive a greater dose after categorization. In some embodiments, Non-Responders and/or Relapsers receive a smaller dose after categorization. In some embodiments, Non-Responders and/or Relapsers receive the same dose after categorization. In some embodiments, Non-Responders and/or Relapsers receive substantially the same dose after categorization.

In some embodiments, the patient is classified as a responder if the pharmaceutical compositions disclosed herein are administered in an induction phase and a maintenance phase, and during the induction phase no improvement in Peak eosinophilic counts in at least one esophageal biopsy and/or worsening of patient mean assessment scores are observed, and where the maintenance phase comprises a dose at least equal to, more than or less than the induction phase.

In some embodiments, the present disclosure provides methods for assessing the suitability of subjects for treatment of EoE. In some embodiments, the recruitment of subjects into the clinical trial is assessed on patient's eosinophil count and score on the PRO assessment that falls within the treatment range (as described herein), prior to treatment. In some embodiments, the patients selected for the clinical trial have Peak eosinophil counts per HPF of greater than about 6, greater than about 10, greater than about 15, or greater than about 20, and more than 6 episodes of dysphagia per week over a time period of two weeks. In some embodiments, the patients selected for the clinical trial have Peak eosinophil counts per HPF of greater than about 15, and more than 6 episodes of dysphagia per week over a time period of two weeks. In some embodiments, the patients selected for the clinical trial have failed prior treatment. In some embodiments, the prior treatment was administration of a PPI over at least about 8 weeks that had not been effective to substantially improve one or more symptoms of EoE.

In addition to Eoe, method disclosed herein may be used to treat, monitor, diagnose, etc, inflammatory conditions of the gastrointestinal tract, including but not limited to inflammation of the esophagus, inflammation of the glottis, inflammation of the epiglottis, inflammation of the tonsils, inflammation of the oropharynx, eosinophilic esophagitis (EoE), gastroesophageal reflux disease (GERD), non-erosive reflux disease (NERD), erosive esophagitis, Barrett's esophagus, eosinophilic gastroenteritis, hypereosinophilic syndrome, corrosive (caustic) chemical esophagitis, radiation-induced esophagitis, chemotherapy-induced esophagitis, transient drug-induced esophagitis (also known as medication esophagitis), persistent drug-induced esophagitis, Crohn's disease of the esophagus, and pseudomembranous esophagitis.

### Device

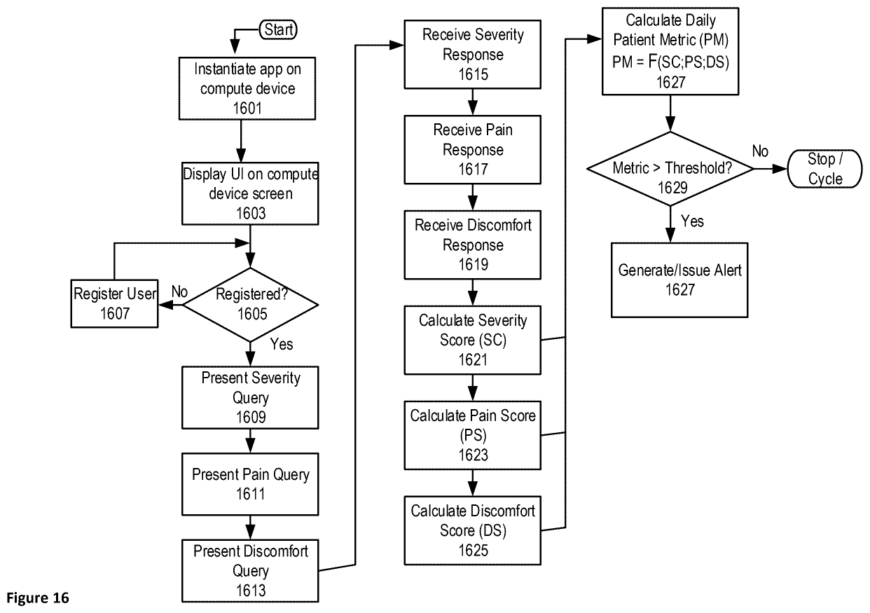

As illustrated by the logic flow in FIG. 16, embodiments of the disclosure include a method for assessing and/or treating dysphagia, the method comprising: instantiating a dysphasia treatment application (1601) on a mobile compute device (such as, by way of non-limiting example, smartphone or tablet), the dysphasia treatment application including a user interface (1603) presented on a display of the mobile compute device. Then a user of the dysphasia treatment application is validated/authenticated (1605), e.g., login credentials/password and/or other HIPAA compliant authentication methods/techniques), or if the user is not validated/registered (1605), registering/validating a user for the first time (1607). A plurality of queries regarding episode-based dysphagia events for a patient is presented via the user interface (1609, 1611, 1613), the user interface being configured to receive user responses. The application receives user responses to the questionnaire, the user responses including: (i) at least one response regarding a severity of a dysphagia event for the patient (1615), (ii) at least one response regarding pain associated with the dysphagia event for the patient (1617), and (iii) at least one response regarding discomfort associated with the dysphagia event for the patient (1619). Then a score for a specified period (e.g., 21 days) can be calculated, the calculation including: (1) determining a severity score (1621) based on the at least one response regarding severity, (2) determining a pain score (1623) based on the at least one response regarding pain, and (3) determining a discomfort score (1625) based on the at least one response regarding discomfort. Depending on the embodiment, the determined score can be, by way of non-limiting example, a binary value (e.g., 0 or 1), an integer value (e.g., from 0 to 10), and/or the like. Then, a patient metric (e.g., daily patient metric) can be determined (1627), such as, by way of non-limiting example, via programmatic logic and/or one or more algorithms, models, data analytics, etc., where the patient metric (PM) is a function of the collected responses, e.g., a function of at least the determined severity score (1621), the determined pain score (1623), and the determined discomfort score (1625). The determined patient metric can then be evaluated (1629), such as against a threshold, range, average, limit, parameter, etc., and an alert (1631) is issued (and/or other notification given) if the daily patient metric exceeds the threshold, falls within a treatment range, or is otherwise determined to be actionable. In some embodiments the alert can include an instruction for administration of a therapeutic agent to the patient. In some embodiments, the alert can include a notification to a physician. In some embodiments, the alert can be used for diagnostics and/or treatment planning.

In some embodiments, the disclosure provides a non-transitory computer readable storage medium storing processor-executable instructions that, when executed by one or more processors cause the one or more processors to: (a) instantiate a dysphasia treatment application on a user compute device, the dysphasia treatment application including a user interface to communicate with a user associated with the user compute device; (b) provide an episode-based dysphagia event data questionnaire via the user interface, the user interface having at least one input configured to receive user input, said user input, the episode-based dysphagia event data questionnaire including: (i) at least one query regarding a severity of a dysphagia even for the patient; (ii) at least one query regarding pain associated with the dysphagia event for the patient; and (iii) at least one query regarding discomfort associated with the dysphagia event for the patient;(c) receive responses to the questionnaire via the user interface; (d) calculate a score over a specified period, the calculation including instruction to: (1) determine a severity score based on responses to the at least one query regarding severity (in some embodiments, the determined severity score is from 0 to 10); (2) determine a pain score based on responses to the at least one query regarding pain (in some embodiments, the determined pain score is from 0 to 10); (3) determine a discomfort score based on responses to the at least one query regarding discomfort (in some embodiments, the determined discomfort score is from 0 to 10); (4) determine a sub-period (e.g., daily) patient metric, the patient metric being a function of at least two or more of the severity score, pain score, and discomfort score (in some embodiments, determining the patient metric can include, but is not limited to a summation of the scores, an average of the scores, a weighted average of the scores, etc.); (e) evaluate the sub-period patient metric (e.g., against a threshold, against a rolling average of prior sub-period metrics, within a treatment range, etc.); and (f) issue an alert based on the evaluation of the sub-period patient metric (e.g., if the if the daily patient metric exceeds or is below a threshold, outside of a treatment range, etc.).

In some embodiments, the disclosure includes an apparatus, comprising: one or more processors; a memory having computer-executable instructions and in communication with the one or more processors; and a display in communication with the one or more processors and/or the memory, where upon execution of the computer-executable instructions by the one or more processors, the one or more processors configured to: (a) instantiate a dysphasia treatment application, the dysphasia treatment application including a graphical user interface that is presented on the display; (b) provide a questionnaire via the graphical user interface, the graphical user interface configured to receive user responses, said questionnaire including queries regarding episode-based dysphagia events for a patient, including: (i) at least one query regarding a severity of a dysphagia even for the patient; (ii) at least one query regarding pain associated with the dysphagia event for the patient; and (iii) at least one query regarding discomfort associated with the dysphagia event for the patient; (c) receive responses to the queries; (d) calculate a score over a specified period, the calculation including instructions to: (1) determine a severity score based on responses to the at least one query regarding severity (in some embodiments, the determined severity score is from 0 to 10); (2) determine a pain score based on responses to the at least one query regarding pain (in some embodiments, the determined pain score is from 0 to 10); (3) determine a discomfort score based on responses to the at least one query regarding discomfort (in some embodiments, the determined discomfort score is from 0 to 10); (4) determine a sub-period (e.g., daily) patient metric, the patient metric being a function of at least the severity score, pain score, and discomfort score (in some embodiments, determining the patient metric can include, but is not limited to a summation of the scores, an average of the scores, a weighted average of the scores, etc.); (e) evaluate the sub-period patient metric (e.g., against a threshold, against a rolling average of prior sub-period metrics, within a treatment range, etc.); and (f) issue an alert based on the evaluation of the sub-period patient metric (e.g., if the if the daily patient metric exceeds or is below a threshold, outside of a range, etc.).

According to some embodiments, the disclosure include a software application that runs on a computer (laptop, desktop, smartphone, tablet, server, terminal, virtual machine, etc.) configured to communicate (e.g., transmit, broadcast, etc.) data from the application to a centralized server, repository, and/or database for storage and/or additional processing/analysis. Depending on the embodiment, calculations, processing, and/or analysis can be done by the application (e.g., on a user compute device) and/or done on a server, network, etc. (e.g., in a distributed manner). Embodiments include security protocols to ensure data integrity and privacy (e.g., compliant with HIPAA, etc.).

A variety of communication protocols can be utilized, including but not limited to Transmission Control Protocol/Internet Protocol (TCP/IP), Hypertext Transfer Protocol Secure (HTTPS), Hypertext Transfer Protocol (HTTP), File Transfer Protocol (FTP), Secure Shell (SSH), POP, Secure Socket Layer (SSL), IMAP, and combinations thereof to transmit information.

In some embodiments, the application can include an alert or reminder tool that can notify a user about determination, request responses, and/or provide information to user. For example, in an embodiment where the application is instantiated on a smart phone, the tool can include visual and/or audio alerts, via the application directly (e.g., notification on screen) and/or via other hardware of the compute device (LED, buzzer, etc.). The above-described embodiments can be implemented in any of numerous ways. For example, the embodiments can be implemented using hardware, software, or a combination thereof. When implemented in software, the software code can be executed on any suitable processor or collection of processors, whether provided in a single computer or distributed among multiple computers.

Further, it should be appreciated that embodiments of the disclosure can be used with and/or on a computer, which can be embodied in any of a number of forms, such as a rack-mounted computer, a desktop computer, a laptop computer, or a tablet computer. Additionally, a computer can be embedded in a device not generally regarded as a computer but with suitable processing capabilities, including a Personal Digital Assistant (PDA), a smart phone or any other suitable portable or fixed electronic device. A computer can have one or more input and output devices, including one or more displays. These input and output devices can be used to present a user interface. Examples of output devices that can be used to provide a user interface include display screens for visual presentation of output and speakers or other sound generating devices for audible presentation of output. Examples of input devices that can be used for a user interface include keyboards, and pointing devices, such as mice, touch screens, and styluses. As another example, a computer can receive input information through speech recognition or in other audible format.

Such computers can be interconnected by one or more networks in any suitable form, including a local area network or a wide area network, such as an enterprise network, and intelligent network (IN) or the Internet. Such networks can be based on any suitable technology and can operate according to any suitable protocol and can include wireless networks, wired networks or fiber optic networks.

The various methods or processes outlined herein can be coded as software that is executable on one or more processors that employ any one of a variety of operating systems or platforms. Additionally, such software can be written using any of a number of suitable programming languages and/or programming or scripting tools, and also can be compiled as executable machine language code or intermediate code that is executed on a framework or virtual machine.

In this respect, various inventive concepts can be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the disclosure discussed above. The computer readable medium or media can be transportable, such that the program or programs stored thereon can be loaded onto one or more different computers or other processors to implement various aspects of the present disclosure as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but can be distributed in a modular fashion amongst a number of different computers or processors to implement various aspects of the present disclosure.

Processor-executable instructions can be in many forms, such as program modules, executed by one or more compute devices, and can include routines, programs, objects, components, data structures, etc. that perform particular tasks or implement particular data types, and the functionality can be combined and/or distributed as appropriate for various embodiments. Data structures can be stored in processor-readable media in a number of suitable forms. For simplicity of illustration, data structures can be shown to have fields that are related through location in the data structure. Such relationships can likewise be achieved by assigning storage for the fields with locations in a processor-readable medium that conveys relationship(s0 between the fields. However, any suitable mechanism/tool can be used to establish a relationship between information in fields of a data structure, including through the use of pointers, tags or other mechanisms/tools that establish relationship between data elements.

Various disclosed concepts can be embodied as one or more methods, of which examples have been provided. The acts performed as part of a particular method can be ordered in any suitable way. Accordingly, embodiments can be constructed in which acts are performed in an order different than illustrated/discussed, which can include performing some acts simultaneously, even though shown as sequential acts in illustrative embodiments.

The use of flow diagrams is not meant to be limiting with respect to the order of operations performed. The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

### EXAMPLES

### Example 1—PROSE Instrument

The data for the PRO assessment (also described herein as the PROSE Instrument) was collected over a 28-day baseline. Item data was summarized over two 14-day periods, days −28 to −15, and days −14 to −1 by producing counts of identification items (e.g. number of episodes, source of issue and remedy) or by taking the average of ratings (e.g., difficulty, pain and discomfort). Baseline was defined as the 14-day period immediately preceding randomization day (i.e., study days −14 through −1). The Week 12 assessments were defined as the 14-day period immediately preceding Week 12 visit (i.e., study days 71 through 84). Change from baseline to Week 12 for all PROSE scores were defined as the Week 12 value minus the baseline value.

The following composite of single question scores were computed:
Number of different food types consumed over 24 h were zero if subjects reported that they did not have any of the items in the past 24 hours. Otherwise, the count of the items endorsed was computed.

The following summary items scored for each valid day of reporting from episode records plus additional items at end of day entry was computed as follows:
Number of RTE (real-time episode entry) dysphagia episodes over the 24-hour period was the count of the number of real-time episodes captured by the device.
Number of EOD (end of day recorded) dysphagia episodes over the 24-hour period was the count of the number of episodes reported as not being captured in the RTE records
Total number of dysphagia episodes over the 24-hour period was the count of the number of real-time episodes plus the number of episodes reported as not being captured by the device.
The proportion of RTE dysphagia episodes over the 24-hour period was the count of the number of real-time episodes captured by the device divided by the total number of dysphagia episodes over the 24-hour period.
Total duration of dysphagia (sum of times for all real-time recorded episodes). Duration was reported in minutes with episodes identified as lasting less than a minute being scored as 0.5 minutes and episodes recorded in hours being scored as the number of hours multiplied by 60.
Total imputed duration of dysphagia. Each EOD recorded episode was assigned the median duration of all RTE for the patient. The imputed duration was the sum of times for all real-time recorded episodes plus the sum of the imputed EOD episodes. Duration was reported in minutes with episodes identified as lasting less than a minute being scored as 0.5 minutes and episodes recorded in hours being scored as the number of hours multiplied by 60.
The number of dysphagia free days. A day was identified as a dysphagia free day if no RTE are reported and the patient responded “No” to the following EOD question:
   In the last 24 hours, did you have any difficulty with food or pills going down that you did NOT record?
Worst difficulty recorded in an RT episode over the 24-hour period was computed as the maximum reported difficulty response among the RT episodes recorded.
Worst pain recorded in an RT episode over the 24-hour period was computed as the maximum reported pain response among the RT episodes recorded.
Worst discomfort recorded in an RT episode over the 24-hour period was computed as the maximum reported discomfort response among the RT episodes recorded.
Worst composite symptom summary score in an RT episode over the 24-hour period was computed as the maximum reported average of the difficulty, pain, and discomfort from each RT episode recorded.
Worst difficulty recorded in an EOD episode over the 24-hour period was computed as the maximum reported difficulty response among the EOD episodes recorded.
Worst pain recorded in an EOD episode over the 24-hour period was computed as the maximum reported pain response among the EOD episodes recorded.
Worst discomfort recorded in an EOD episode over the 24-hour period was computed as the maximum reported discomfort response among the EOD episodes recorded.
Worst composite symptom summary score in an EOD episode over the 24-hour period was computed as the maximum reported average of the difficulty, pain, and discomfort from each EOD episode recorded.
Worst difficulty recorded in any episode during day was computed as the maximum reported difficulty response.
Worst pain recorded in any episode during day was computed as the maximum reported pain response.
Worst discomfort recorded in any episode during the day was computed as the maximum reported discomfort response.
Worst composite symptom summary score in any episode over the 24-hour period was computed as the maximum reported average of the difficulty, pain, and discomfort from each episode recorded.

For each of these items the mean score was taken over the 14-day assessment period. Subjects must have had at least eight days of PROSE data to be included. Subjects with less than eight valid days of reporting were excluded from analyses.

Additionally, the average of all ratings over each 14-day period were computed as follows:
The average difficulty recorded on all RT episodes occurring on valid days over each 14-day period.
The average pain recorded on all RT episodes occurring on valid days over each 14-day period.
The average discomfort recorded on all RT episodes occurring on valid days over each 14-day period.
The average difficulty recorded on all EOD episodes occurring on valid days over each 14-day period.
The average pain recorded on all EOD episodes occurring on valid days over each 14-day period.
The average discomfort recorded on all EOD episodes occurring on valid days over each 14-day period.
The average difficulty recorded on all episodes occurring on valid days over each 14-day period.
The average pain recorded on all episodes occurring on valid days over each 14-day period.
The average discomfort recorded on all episodes occurring on valid days over each 14-day period.
The average difficulty recorded on all 24 h records for each 14-day period.
The average pain recorded on all 24 h records for each 14-day period.
The average discomfort recorded on all 24 h records for each 14-day period.

Thus, the following scores summarizing data from the 14-day period were included in the measurement properties (MP) analysis:
Worst difficulty from 24 h questions (WDF24; 1 item; range 0-10).
Worst pain from 24 h questions (WPN24; 1 item; range 0-10).
Worst discomfort from 24 h questions (WDC24; 1 item; range 0-10).
Average composite symptom summary score from all 24 h questions (RATESUM24; 1 item; 0-10 range).
Worst difficulty from episode record (WDFEV; 1 item; range 0-10).
Worst pain from episode record (WPNEV; 1 item; range 0-10).
Worst discomfort from episode record (WDCEV; 1 item; range 0-10).
Worst composite symptom summary score from episode record (RATESUMEV; 1 item; 0-10 range).
Average difficulty from all episode records occurring on valid days (WDFEVPER; 1 item; range 0-10).
Average pain from all episode records occurring on valid days (WPNEVPER; 1 item; range 0-10).
Average discomfort from all episode records occurring on valid days (WDCEVPER; 1 item; range 0-10).
Average composite symptom summary score from all episode records occurring on valid days (RATESUMPER; 1 item; 0-10 range).
Number of dysphagia episodes (DSNUM; 1 item summed over a 24-hour period, then the mean taken over the valid days).
Total duration of dysphagia (DSDUR; 1 item summed over a 24-hour period; range 0-24 hours, then the mean taken over the valid days).
Number of food types consumed (FTNUM; multiple items summed over a 24-hour period, range 0-6, then the mean taken over the valid days).
Number of dysphagia free days (VNONEPDY, range 0-14).

Compliance with the PROSE was calculated as number of days during the 14-day baseline period in which a valid entry was made. A valid entry was defined as completing the EOD record. FIGS. 3-15 detail results using this instrument.

### Example 2—Co-Primary Endpoints for EoE Using the PROSE Instrument

Recommended endpoints for treatment of EoE include co-primary endpoints that measure symptom improvements and histological endpoints. Using the PROSE questionnaire, the following endpoints were assessed:

The average episode symptom summary rating. This is the average over all episodes of the 14 day mean of three symptom ratings based on the following questions:
1. How difficult was it for you to get the [food/pills] down?
2. What was the worst pain you felt when trying to get the [food/pills] down?
3. What was the worst discomfort you felt when trying to get the [food/pills] down?

The maximum episode summary rating. This is the average of the maximum daily mean over 14 days of three symptom ratings based on the following questions:
1. How difficult was it for you to get the [food/pills] down?
2. What was the worst pain you felt when trying to get the [food/pills] down?
3. What was the worst discomfort you felt when trying to get the [food/pills] down?

The 24-hour worst symptom summary rating. This is the average of the three evening diary (i.e. 24-hour summary) based on the following questions.
1. In the last 24 hours, what was the worst difficulty you experienced when trying to get food or pills down?
2. In the last 24 hours, what was the worst pain you experienced when trying to get food or pills down?
3. In the last 24 hours, what was the worst discomfort you experienced when trying to get food or pills down?

Dysphagia Free Days. This is the number of days with no dysphagic episode. A day will be defined as a dysphagia free day if no real time episodes (RTE) are reported and the patient responds “no” to the following Evening Diary question:
1. In the last 24 hours, did you have any difficulty with food or pills going down that you did NOT record using the “Report difficulty with food or pills going down” button?

Total Dysphagia Episodes. This is the total number of episodes recorded over the observation period. The total number of dysphagia episodes over the period will be the count of the number of real-time episodes plus the number of episodes reported via the end of day catch.

Table 1 shows co-primary endpoints for use with the PROSE instrument.

TABLE 1

| | | | | | Baseline/wk | | |
|---|---|---|---|---|---|---|---|
| | | | | Baseline/wk | 12 (change)^{†}- | | |
| | | | | 12 (change)^{†}- | for PGIC | Baseline/wk 12 | Baseline/wk 12 |
| | | | | for PGIC | symptom | (change)^{†}-for | (change)^{†}-for |
| Endpoint | | | Psycho- | symptom | non- | PGIC difficulty | PGIC difficulty non- |
| Consideration | Description | Qualitative | metrics | improvers | improvers | improvers | improvers |
| Episode | Average | Summary | √ | 3.9/3.0 (24%) | 4.7/4.8 | 4.0/3.1 (23%) | 4.6/4.7 |
| Average | over all | ratings | | | (−3%) | | (−4%) |
| Symptom | episodes of | include some | | | | | |
| Summary | the mean of | of the most | | | | | |
| Rating | 3 symptom | commonly | | | | | |
| | ratings | mentioned | | | | | |
| Episode Max | Maximum | characteristics | √ | 4.2/3.0 (28%) | 4.8/4.8 (0%) | 4.3/3.1 (26%) | 4.7/4.8 |
| Summary | daily value | of dysphagia | | | | | −1%) |
| Rating | of the mean | episodes; very | | | | | |
| | of 3 | meaningful to | | | | | |
| | symptom | patient | | | | | |
| | ratings | experience | | | | | |
| 24 h Worst | Average of | | √ | 3.6/1.9 (47%) | 4.2/4.1 (4%) | 3.6/2.0 (45%) | 4.1/4.0 (2%) |
| Symptom | the mean of | | | | | | |
| Summary | 3 24 h | | | | | | |
| Rating | symptom | | | | | | |
| | ratings | | | | | | |
| Dysphagia | Number of | | √ | 3.4/7.4 (115%) | 3.9/4.8 (21%) | 3.4/7.2 (112%) | 4.2/5.0 (18%) |
| Free Days | days with | | | | | | |
| | no (0) | | | | | | |
| | episodes | | | | | | |
| Total | Total | | √ | 15.2/6.4 (58%) | 12.8/9.9 (23% | 15.1/6.5 (57%) | 12.5/10.3 (18%) |
| Dysphagia | number of | | | | | | |
| Episodes | episodes | | | | | | |
| | recorded | | | | | | |
| | over | | | | | | |
| | observation | | | | | | |
| | period | | | | | | |

Symptoms: Difficulty, Pain and Discomfort
^{†}Values shown are mean and percent change;
Percent change defined as (Baseline Mean-Week 12 Mean)/Baseline Mean;
Positive values indicate improvement
PGIC = patient global impression of change

The analysis of the data in Table 1 was blinded, as some patients from the placebo arm of the clinical trial were included in the analysis. Thus, the actual results for patients that were treated with a corticosteroid will show a higher percent change from baseline.

### INCORPORATION BY REFERENCE

All publications, patents, and patent publications cited are incorporated by reference herein in their entirety for all purposes.

This application incorporates by reference the following publications and applications in their entireties for all purposes: PCT/US2017/047474, filed Aug. 17, 2017, U.S. Appln. No. 62/376,703, filed Aug. 18, 2016, U.S. Appln. No. 62/461,317, filed Feb. 21, 2017, U.S. Appln. No. 62/489,292, filed Apr. 24, 2017, U.S. Pat. No. 8,771,729 filed Oct. 1, 2010; US 2016/0206627 filed Sep. 5, 2014, U.S. 61/874,450 filed Sep. 6, 2013, WO 2015/034678 filed Aug. 21, 2014, and WO 2015/035114 filed Sep. 5, 2014.

1. A method of managing eosinophilic esophagitis (EoE) in a patient in need thereof, comprising:
(i) prior to treatment with a therapeutic agent,
(a) providing, via a digital processing device, a patient-reported outcome (PRO) questionnaire;
(b) recording each episode of dysphagia, at the time the episode occurs, for a period of at least about two weeks using the PRO questionnaire; and
(c) measuring esophageal eosinophils in the patient; then
(ii) treating the patient with a therapeutically effective amount of a therapeutic agent for at least two weeks; and
(iii) recording, using the PRO questionnaire, each episode of dysphagia, at the time each episode occurs, while the patient is being treated,
wherein dysphagia over a two week period of time while the patient is being treated is reduced compared to the dysphagia prior to treatment.

2. The method of claim 1, wherein recording in step (i)(b) comprises recording one or more of:
incidence of an episode of dysphagia;
duration of dysphagia,
severity of dysphagia
pain caused by dysphagia;
discomfort of dysphagia;

3. The method of claim 1, wherein recording in step (iii) comprises recording one or more of:
incidence of an episode of dysphagia,
duration of dysphagia,
severity of the episode of dysphagia,
time and date of administering treatment.

4. The method of claim 1, wherein the therapeutic agent is a corticosteroid, a proton pump inhibitor (PPI), or an antibody.

5. The method of claim 4, wherein the corticosteroid is budesonide, fluticasone, flunisolide, ciclesonide, mometasone, beclomethasone, or tixocortol, or a salt, ester, solvate, polymorph, or prodrug thereof.

6. The method of claim 4, wherein the PPI is omeprazole, lansoprazole, dexlansoprazole, esomeprazole, pantoprazole, or rabeprazole

7. The method of claim 4, wherein the antibody is an IL-4, IL-5, or IL-13 antibody.

8. The method of claim 7, wherein the antibody is benralizumab, mepolizumab, dupilumab, RPC-4046.

9. The method of claim 1, wherein the esophageal eosinophils are measured by obtaining a biopsy.

10. The method of claim 9, wherein the biopsy is an endoscopy.

11. The method of claim 1, wherein the patient has an esophageal eosinophil count of ≥15 per high-power field (HPF).

12. The method of claim 1, further comprising measuring esophageal eosinophils in the patient after the patient has been treated with the therapeutic agent for at least two weeks.

13. The method of claim 11, wherein the patient is a histological non-responder.

14. The method of claim 12, wherein the patient has an esophageal eosinophil count of ≤15 per high-power field (HPF).

15. (canceled)

16. (canceled)

17. The method of claim 1, wherein after step (iii) the patient continues treatment with the therapeutic agent at the same dose as used in step (ii).

18. The method of claim 1, after step (iii), the method further comprises administering a dose of the therapeutic agent which is decreased by at least about 5%.

19. The method of claim 18, wherein the patient is treated with the decreased dose of the therapeutic agent for at least the period of time during which the number of episodes of dysphagia are reduced as determined via the PRO questionnaire.

20. The method of claim 19, wherein if the number of episodes of dysphagia increases while the patient is receiving the decreased dose, as determined via the PRO questionnaire, the method further comprises administering the same dose as in step (ii).

21-57. (canceled)

58. A non-transitory computer readable storage media device encoded with a computer program including instructions executable by a digital processing device for treating dysphagia in a patient in need thereof, comprising
(a) instructions configured to provide a questionnaire to a patient, wherein the questionnaire comprises: at least one input to record episode-based dysphagia events; wherein said input records:
(i) at least one question determining the severity of the dysphagia event as the event occurs;
(ii) at least one question determining the pain associated with the dysphagia event, as the event occurs; and
(ii) at least one question determining the discomfort associated with the dysphagia event, as the event occurs; and
(b) instructions configured to apply via the digital processing device an algorithm to answers to said questions to determine a score calculated over 1-21 days,
wherein the algorithm comprises:
scoring the at least one severity question from 0 to 10;
scoring the at least one pain question from 0 to 10;
scoring the at least one discomfort question from 0 to 10;
summing the scores of all the questions presented in the questionnaire; and
calculating the daily average score;
(c) evaluating the evaluating the daily score against a treatment range; and
(d) if the daily score falls within a treatment range, the device is configured to instruct the administration of a therapeutic agent.

59-74. (canceled)

75. A method for treating dysphagia in a patient in need thereof, comprising
(a) providing, via a digital processing device, a PRO questionnaire to a patient, wherein the PRO questionnaire comprises:
(i) at least one question determining the severity of the dysphagia event, as the event occurs;
(ii) at least one question determining the pain associated with the dysphagia event, as the event occurs; and
(ii) at least one question determining the discomfort associated with the dysphagia event, as the event occurs; and
(b) applying, via the digital processing device, an algorithm to answers to said questions to determine a score calculated over 1-21 days,
wherein the algorithm comprises:
scoring the at least one severity question from 0 to 10;
scoring the at least one pain question from 0 to 10;
scoring the at least one discomfort question from 0 to 10;
summing the scores of all the questions presented in the questionnaire; and
calculating the daily average score;
(c) evaluating the evaluating the daily score against a treatment range; and
(d) administering a therapeutic agent to the patient when the daily average score falls within the treatment range.

76-93. (canceled)
